(19) United States
(12) Patent Application Publication
(10) Pub. No.: US 2024/0210419 A1
(43) Pub. Date: Jun. 27, 2024

(54) COMPOSITIONS AND METHODS FOR DETECTING AND REGULATING FIBRONECTIN-INTEGRIN INTERACTIONS AND SIGNALING

(71) Applicant: University of Virginia Patent Foundation, Charlottesville, VA (US)

(72) Inventors: Thomas H. Barker, Charlottesville, VA (US); Jagathpala Shetty, Charlottesville, VA (US)

(73) Assignee: University of Virginia Patent Foundation, Charlottesville, VA (US)

(21) Appl. No.: 18/573,975

(22) PCT Filed: Jun. 22, 2022
(86) PCT No.: PCT/US2022/034456

§ 371 Date: Dec. 22, 2023

Related U.S. Application Data
(60) Provisional application No. 63/213,492, filed on Jun. 22, 2021.

(51) Int. Cl. G01N 33/68; A61K 39/00; C07K 16/18

(52) U.S. Cl. CPC G01N 33/6857; C07K 16/18; G01N 33/6887; A61K 2039/505; G01N 2800/10; G01N 2800/52

(57) ABSTRACT

Provided are antibodies that include amino acid sequences of SEQ ID NOs: 2, 4, 6, 14, 16, or 18, or amino acid sequences that are about 95% identical thereto, and paratop-containing fragments thereof. Also provided are nucleic acids encoding a VII segment comprising, consisting essentially of, or consisting of SEQ ID NO: 4, a VL segment comprising, consisting essentially of, or consisting of SEQ ID NO: 16, or a combination thereof: methods for using the same to detect and/or target conformational states of FN in samples: methods for treating diseases and/or disorders and/or for meliorating at least one symptom of consequence of a disease or disorder associated with abnormal expression of a force-induced conformational state of FN in subjects: and methods for screening for compounds having selective binding activities for conformational states of FN.

### CROSS REFERENCE TO RELATED APPLICATION

The presently disclosed subject matter claims the benefit of U.S. Provisional Patent Application Ser. No. 63/213,492, filed Jun. 22, 2021, the disclosure of which incorporated herein by reference in its entirety.

### REFERENCE TO SEQUENCE LISTING SUBMITTED ELECTRONICALLY

The content of the electronically submitted sequence listing in ASCII text file (Name: 3062_161_PCT_ST25.txt; Size: 29 kilobytes; and Date of Creation: Jun. 22, 2022) filed with the instant application is incorporated herein by reference in its entirety.

### TECHNICAL FIELD

The presently disclosed subject matter relates to compositions comprising modified antibodies and fragments thereof, and methods for using the same for detecting and regulating fibronectin-integrin interaction and signaling. In particular, the presently disclosed subject matter relates to compositions and methods useful for targeting of a mechanically exposed cryptic site within fibronectin's integrin binding domain.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter relates to provides isolated and purified antibodies that comprise, consist essentially of, or consist of an amino acid sequence selected from the group consisting of SEQ ID NOs: 2, 4, 6, 14, 16, or 18, or fragments thereof, or antibodies having an amino acid sequence that is approximately 95% identical to one of SEQ ID NOs: 2, 4, 6, 14, 16, or 18. In some embodiments, the antibody or the fragment thereof is humanized.

In some embodiments, the isolated and purified antibody or the fragment thereof comprises a heavy chain CDR1 comprising the amino acid sequence SYAMS (SEQ ID NO: 8), a heavy chain CDR2 comprising the amino acid sequence DIYDGGGTNYADSVKG (SEQ ID NO: 10), a heavy chain CDR3 comprising the amino acid sequence TADNFDY (SEQ ID NO: 12), a light chain CDR1 comprising the amino acid sequence RASQSISSYLN (SEQ ID NO: 20), a light chain CDR2 comprising the amino acid sequence AASTLQS (SEQ ID NO: 22), and a light chain CDR3 comprising the amino acid sequence QQANSAPTT (SEQ ID NO: 24); and/or the isolated and purified antibody further comprises a heavy chain framework region 1 comprising EVQLLESGGGLVQPGGSLRLSCAAS (SEQ ID NO: 40), a heavy chain framework region 2 comprising WVRQAPGKGLEWV (SEQ ID NO: 41), a heavy chain framework region 3 comprising RFTTSRDNSKNTLYLQMNSLRAEDTAVYYC (SEQ ID NO: 42), and a heavy chain framework region 4 comprising WGQGTLVTVSS (SEQ ID NO: 43); and/or the isolated and purified antibody further comprises a light chain framework region 1 comprising DIQMTQSPSSLSASVGDRVTITC (SEQ ID NO: 44), a light chain framework region 2 comprising WYQQKPGKAPKLLIY (SEQ ID NO: 45), a light chain framework region 3 comprising GVPSRFSGSGSGTDFTLTISSLQPEDFATYYC (SEQ ID NO: 46), and a light chain framework region 4 comprising FGQGTKVEIK (SEQ ID NO: 47).

In some embodiments, the isolated and purified antibody, or fragment or homolog thereof, comprises a modification at its N-terminus, its C-terminus, or both. In some embodiments, the modification comprises addition of a peptide tag, a SARAH domain, or a combination thereof. In some embodiments, the tag comprises a His tag (e.g., HHHHHH; SEQ ID NO: 35), a myc tag (e.g., EQKLISEEDL; SEQ ID NO: 33), a VSV tag (e.g., YTDIEMNRLGK; SEQ ID NO: 34), an HA tag (e.g., YPYDVPDYA; SEQ ID NO: 36), a SortaseA tag (e.g., LPTEGG (SEQ ID NO: 37) and/or LPXTG; SEQ ID NO: 48), a PelB sequence (e.g., MKYLLPTAAAGLLLLAAQPAMA (SEQ ID NO: 38) or MKYLLPTAEAGLLLLLAAPQIA (SEQ ID NO: 49)), or any combination of one or more thereof. In some embodiments, the SARAH domain comprises a sequence selected from the group consisting of SEQ ID NOs: 28-32.

The presently disclosed subject matter also provides in some embodiments isolated and purified nucleic acid sequences encoding the antibodies and fragments disclosed herein.

The presently disclosed subject matter also provides in some embodiments methods for targeting conformational states of fibronectin (FN) in samples, optionally biological samples isolated from or present within a subject. In some embodiments, the methods comprise contacting a sample with a composition having a selective binding activity for a conformational state of FN comprising FnIII9-4G-10 (4G), whereby the conformational state is targeted. In some embodiments, the sample comprises or is suspected to comprise a tissue undergoing tissue repair, a tissue that is diseased, a tissue that suffers from a disorder, or any combination thereof.

The presently disclosed subject matter also provides in some embodiments methods for detecting conformational states of fibronectin (FN) in samples. In some embodiments, the methods comprise contacting a sample with a composition having a selective binding activity for a conformational state of FN comprising FnIII9-4G-10 (4G); and detecting the binding of the composition, whereby the conformational state of FN is detected. In some embodiments, the sample comprises or is suspected to comprise a tissue undergoing tissue repair, a tissue that is diseased, a tissue that suffers from a disorder, or a combination thereof. In some embodiments, the sample comprises or is suspected to comprise a pathologic extracellular matrix (ECM). In some embodiments, the sample comprises or is suspected to comprise a fibrotic ECM. In some embodiments, detecting the binding of the composition comprises detecting a binding ratio of composition to FN. In some embodiments, detecting the binding of the composition comprises istinguishing normal from diseased tissue. In some embodiments, detecting the binding of the composition comprises determining severity of fibrosis in the sample. In some embodiments, detecting the binding of the composition comprises detecting a transient, force-induced conformational change in FN. In some embodiments, detecting the binding of the composition comprises extracting structural information for an ECM in the sample. In some embodiments, extracting structural information for an ECM in the sample comprises delineating regions of high ECM strain. In some embodiments, the high ECM strain is associated with enhanced av integrin binding character.

In some embodiments, the methods further comprise determining a type of treatment to be administered to the subject based on the detecting of the binding of the composition.

The presently disclosed subject matter also provides in some embodiments methods for treating diseases and/or disorders in subjects. In some embodiments, the methods comprise administering to a subject in need there of a therapeutically effective amount of a composition having a selective binding activity for a conformational state of FN comprising FnIII9-4G-10 (4G), whereby treatment is accomplished. In some embodiments, the disease and/or disorder has a characteristic selected from the group consisting of a tissue undergoing tissue repair, a tissue that is diseased, a tissue that suffers from a disorder, and any combination thereof. In some embodiments, the characteristic is a pathologic extracellular matrix (ECM). In some embodiments, the characteristic is a fibrotic ECM.

In some embodiments of the presently disclosed methods, the composition having a selective binding activity for a conformational state of FN comprising FnIII9-4G-10 (4G) is an isolated and purified antibody comprising an amino acid sequence selected from the group consisting of SEQ ID NOs: 2, 4, 6, 14, 16, or 18, a fragment thereof, or an antibody have a sequence approximately 95% identical to a sequence of SEQ ID NOs: 2, 4, 6, 14, 16, or 18, or a fragment thereof. In some embodiments, the amino acid sequence comprises at least one modification selected from the group consisting of an amino acid deletion, an amino acid addition, an amino acid substitution, and combinations thereof. In some embodiments, the antibody or the fragment thereof is humanized.

The presently disclosed subject matter also provides in some embodiments methods for screening for antibodies and/or fragments and/or derivatives thereof having selective binding activities for conformational states of FN comprising FnIII9-4G-10 (4G). In some embodiments, the methods comprise providing a sample comprising a conformational state of FN comprising FnIII9-4G-10 (4G); contacting the sample with a candidate antibodies and/or fragments and/or derivatives thereof; and detecting binding of the candidate antibodies and/or fragments and/or derivatives thereof to the sample. In some embodiments, the candidate antibodies and/or fragments and/or derivatives thereof are members of a library of antibodies and/or fragments and/or derivatives thereof. In some embodiments, the candidate antibodies and/or fragments and/or derivatives thereof are intact antibodies. In some embodiments, the conformational state of FN is a force-induced conformational change in Fn.

The presently disclosed subject matter also provides in some embodiments antibodies and/or fragments and/or derivatives thereof identified by the presently disclosed methods.

The presently disclosed subject matter also provides in some embodiments methods for treating diseases and/or disorder in subjects comprising administering to a subject in need thereof a therapeutically effective amount of a composition comprising the antibody or fragment thereof in accordance with the presently disclosed subject matter, whereby treatment is accomplished.

The presently disclosed subject matter also provides in some embodiments methods for ameliorating at least one symptom of consequence of a disease or disorder associated with abnormal expression of a force-induced conformational state of FN comprising FnIII9-4G-10 (4G) in a subject. In some embodiments, the methods comprise administering to a subject in need thereof a therapeutically effective amount of a composition comprising an antibody or fragment thereof in accordance with the presently disclosed subject matter, wherein at least one symptom of consequence of a disease or disorder associated with abnormal expression of a force-induced conformational state of FN comprising FnIII9-4G-10 (4G) is ameliorated.

In some embodiments of the therapeutic methods, the disease or disorder is associated with a tissue undergoing tissue repair, a tissue that is diseased, a tissue that suffers from a disorder, or any combination thereof. In some embodiments, the disease or disorder is associated with a pathologic extracellular matrix (ECM). In some embodiments, the disease or disorder is associated with a fibrotic ECM.

Accordingly, it is an object of the presently disclosed subject matter to provide compositions and methods for detecting and regulating fibronectin-integrin interaction and signaling. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following Description, FIGURE, and EXAMPLE. Additionally, various aspects and embodiments of the presently disclosed subject matter are described in further detail below.

### BRIEF DESCRIPTION OF THE FIGURE

FIG. 1 is a graph showing the results of in vitro ELISA binding assays to determine the efficacy of binding of H5 antibodies and derivatives thereof to the diseased form (dise) and the healthy form (norm) of fibronectin (Fn) at various dosages. The data represent the combination of two independent experiments: one comparing E. coli (eH5) versus N. benthamiana (nicoH5) produced H5-scFv and the second comparing H5-scFv (H5(scFv)) to H5-IgG1 (H5(IgG)). eH5 v. Fn (dise): open squares and a heavy black line; eH5 v. Fn (norm): open circle and a thin black line; nicoH5 v. Fn (dise): solid squares and a dashed line with larger dashes; nicoH5 v. Fn (norm): solid circle and a dashed line with smaller dashes; H5(scFv) v. Fn (dise): open triangle and a heavy black line; H5(scFv) v. Fn (norm): inverted open triangle and a dashed line; H5(IgG) v. Fn (dise): solid triangle and a thin dashed line; H5(IgG) v. Fn (norm): inverted solid triangle and a heavy dashed line.

### BRIEF DESCRIPTION OF THE SEQUENCE LISTING

SEQ ID NOs: 1 and 2 are the nucleic acid and amino acid sequences, respectively, of the heavy chain of the exemplary H5-IgG1 antibody of the presently disclosed subject matter.

SEQ ID NOs: 3 and 4 are the nucleic acid and amino acid sequences, respectively, of the heavy chain variable region of the exemplary H5-IgG1 antibody of the presently disclosed subject matter.

SEQ ID NOs: 5 and 6 are the nucleic acid and amino acid sequences, respectively, of the heavy chain constant region of the exemplary H5-IgG1 antibody of the presently disclosed subject matter.

SEQ ID NOs: 7 and 8 are the nucleic acid and amino acid sequences, respectively, of the heavy chain CDR1 of the exemplary H5-IgG1 antibody of the presently disclosed subject matter.

SEQ ID NOs: 9 and 10 are the nucleic acid and amino acid sequences, respectively, of the heavy chain CDR2 of the exemplary H5-IgG1 antibody of the presently disclosed subject matter.

SEQ ID NOs: 11 and 12 are the nucleic acid and amino acid sequences, respectively, of the heavy chain CDR3 of the exemplary H5-IgG1 antibody of the presently disclosed subject matter.

SEQ ID NOs: 13 and 14 are the nucleic acid and amino acid sequences, respectively, of the light chain of the exemplary H5-IgG1 antibody of the presently disclosed subject matter.

SEQ ID NOs: 15 and 16 are the nucleic acid and amino acid sequences, respectively, of the light chain variable region of the exemplary H5-IgG1 antibody of the presently disclosed subject matter.

SEQ ID NOs: 17 and 18 are the nucleic acid and amino acid sequences, respectively, of the light chain constant region of the exemplary H5-IgG1 antibody of the presently disclosed subject matter.

SEQ ID NOs: 19 and 20 are the nucleic acid and amino acid sequences, respectively, of the light chain CDR1 of the exemplary H5-IgG1 antibody of the presently disclosed subject matter.

SEQ ID NOs: 21 and 22 are the nucleic acid and amino acid sequences, respectively, of the light chain CDR2 of the exemplary H5-IgG1 antibody of the presently disclosed subject matter.

SEQ ID NOs: 23 and 24 are the nucleic acid and amino acid sequences, respectively, of the light chain CDR3 of the exemplary H5-IgG1 antibody of the presently disclosed subject matter.

SEQ ID NO: 25 is the amino acid sequence of the pentapeptide motif PHSRN that is found in the 9th type III repeat of fibronectin.

SEQ ID NOs: 26 and 27 are the amino acid sequences of an exemplary tetrapeptide linker consisting of four glycine residues and an exemplary pentapeptide linker consisting of a serine residue followed by four glycine residues. It is noted that to create a linker peptide, one, two, three, or more copies of SEQ ID NO: 26 can be combined (i.e., concatemerized), one, two, three, or more copies of SEQ ID NO: 27 can be combined (i.e., concatemerized), or one, two, three, or more copies of SEQ ID NO: 26 can be combined with one, two, three, or more copies of SEQ ID NO: 27.

SEQ ID NOs: 28-32 are the amino acid sequences of exemplary SARAH domains that can be added to the N-terminus, the C-terminus, or both of an antibody or fragment thereof of the presently disclosed subject matter.

SEQ ID NOs: 33-38 are the amino acid sequences of exemplary tags that can be added to the N-terminus, the C-terminus, or both of an antibody or fragment thereof of the presently disclosed subject matter. SEQ ID NO: 31 is an exemplary myc tag, SEQ ID NO: 32 is an exemplary VSV tag, SEQ ID NO: 33 is an exemplary His tag, SEQ ID NO: 34 is an exemplary HA tag, SEQ ID NO: 35 is an exemplary SortaseA tag, and SEQ ID NO: 36 is an exemplary PelB tag.

SEQ ID NO: 39 is an exemplary linker sequence.

SEQ ID NOs: 40-43 are exemplary heavy chain framework regions 1-4, respectively, that can be employed in the the exemplary H5-IgG1 antibody of the presently disclosed subject matter.

SEQ ID NOs: 44-47 are exemplary light chain framework regions 1-4, respectively, that can be employed in the the exemplary H5-IgG1 antibody of the presently disclosed subject matter.

SEQ ID NOs: 48 and 49 are additional amino acid sequences of various exemplary tags, including a SortaseA tag (e.g., LPXTG; SEQ ID NO: 48) and a PelB sequence (e.g., MKYLLPTAEAGLLLLLAAPQIA; SEQ ID NO: 49)

### DETAILED DESCRIPTION

Fibronectin (Fn) is an extracellular matrix protein that orchestrates complex cell adhesion and signaling through cell surface integrin receptors during tissue development, remodeling, and disease, such as fibrosis. Fn is sensitive to mechanical forces in its tandem type III repeats resulting in extensive molecular elongation. As such, it has long been hypothesized that cell- and tissue-derived forces can activate an “integrin switch” within the critical integrin binding 9th and 10th type III repeats—conferring differential integrin binding specificity leading to differential cell responses. Yet, no direct evidence exists to prove the hypothesis nor demonstrate the physiological existence of the switch. Provided in accordance with the presently disclosed subject matter is direct experimental evidence for the Fn integrin switch both in vitro and ex vivo using an antibody engineered to detect the transient, force-induced conformational change, representing an opportunity for detection and targeting of early molecular signatures of cell contractile forces in tissue repair and disease.

The extracellular matrix (ECM) forms the complex niche of structural elements surrounding cells in vivo. Cells interact with and are instructed by the ECM via cellular structures known as focal adhesions, large protein complexes composed of transmembrane receptors (integrins) and intracellular adaptor proteins that mechanically couple the cell's cytoskeleton to fibrillar ECM proteins such as fibronectin (Fn). Protein-protein interactions within focal adhesions are dynamic; mechanical forces play important roles for focal adhesion maturation and development, as well as for force-sensitive cell signaling via mechanosensory proteins. Recent work has shown that conformations of both intracellular focal adhesion constituents (e.g., vinculin, integrins; see Zhu et al., 2008; Grashoff et al., 2010; Carisey et al., 2013) as well as extracellular components (e.g., Fn; see Smith et al., 2007; Lemmon et al., 2011; Cao et al., 2012) are altered by forces transmitted to and from the ECM. In the latter case, previous work demonstrated that Fn within the ECM exhibits distinct but undefined 20) altered structural states in response to cellular forces both in vitro and in vivo (see Chandler et al., 2011; Cao et al., 2012).

Fn comprises three types of tandem repeating units, each containing two antiparallel β-sheets. Type I and II repeats are structurally stabilized by disulfide bonds, whereas type III repeats are stabilized only by hydrogen bonding and Van der Waals forces, making them sensitive to unfolding due to physiologically relevant forces (see Krammer et al., 1999; Craig et al., 200; Craig et al., 2004; Li et al., 2005; Gee et al., 2008). These findings, when coupled with the active role of Fn's 9^{th} and 10^{th} type III repeats (FnIII9-10) in mediating integrin-specific interactions, inspired the theory that mechanical forces could trigger a “switch” in the integrin-binding profile of Fn (Krammer et al., 1999). Fn-integrin interactions are known to drive critical cell behaviors and are mediated primarily through the canonical and promiscuous integrin binding sequence Arg-Gly-Asp (RGD) within the 10^{th} type III repeat (Ruoslahti & Pierschbacher, 1987). A subset of integrins, including integrin α5β1, is additionally dependent on the sequence motif PHSRN (SEQ ID NO: 25) within the neighboring 9th type III repeat (Aota et al., 1994; Mardon & Grant, 1994; Mould et al., 1997; Garcia et al., 2002). Integrin specificity to Fn can be modulated in vitro by altering the structural stability of the integrin binding domain (i.e. the 9th and 10th type III repeats) via directed mutation (van der Walle et al., 2002) resulting in the regulation of developmentally and pathologically relevant cell differentiation pathways (Martino et al., 2009; Brown et al., 2011), and, importantly, cellular responses to microenvironmental mechanics (e.g., stiffness; Markowski et al., 2012). Despite these findings, the integrin switch theory and its potential relevance to biological processes in vivo remained undefined prior to the presently disclosed subject matter.

Reports have suggested that the relative separation distance between the “synergy” PHSRN sequence (SEQ ID NO: 25) in the 9th Fn type III repeat and the RGD site in the 10th Fn type III repeat is critical for engagement and activation of integrins α5β1 (Martino et al., 2009) and α3β1 (Brown et al., 2015), with an optimal PHSRN (SEQ ID NO: 25)-RGD distance of 3.7 nm for high affinity integrin α5β1 engagement (Craig et al., 2008). Furthermore, recent findings demonstrated that Fn fiber extension decreases cell spreading and adhesion (Hubbard et al., 2016).

The development of conformation-specific antibodies by phage display is well established, as work by Lefkowitz and coworkers have used phage display to isolate a conformation specific Fab to activated B-arrestin-1 (Shukla et al., 2013). Yet, particular challenges of the development of the presently disclosed conformation-specific antibodies were that (1) the conformational change of the integrin binding domain was due to the application of force; (2) the application of force to Fn fibers led to multiple conformational changes along the length of the 440 kDa protein; and (3) the conformational change was highly labile due to the ability of Fn type III repeats to refold in the absence of force. Here, predicted structures from steered molecular dynamics simulations coupled with molecular engineering were utilized to produce a mimetic of the strained integrin binding domain in order to perform phage display to discover the parental H5 clone. It is likely that the two model Fn fragments differ not only in separation between RGD and PHSRN (SEQ ID NO: 25), but also in relative conformational stability. FnIII9*10 is stabilized by a Leu 1408Pro mutation between FnIII9 and FnIII1018, whereas FnIII-4G-10 is separated by a 4-glycine linker between the two domains.

One exemplary application of the modified H5-IgG1 antibodies and fragments thereof of the presently disclosed subject matter is to probe pathologic ECMs, in some embodiments fibrotic ECMs, which contain highly contractile myofibroblasts. Recent reports suggest that αv integrins on myofibroblasts are implicated in fibrogenesis in a broad range of fibrotic diseases, and that pharmacological blockade of αv integrins ameliorates liver and lung fibrosis (Henderson et al., 2013). The presently disclosed subject matter use of the modified H5-IgG1 antibody disclosed herein in the context of idiopathic pulmonary fibrosis (IPF), a fatal form of progressive lung fibrosis in humans. The lungs of IPF patients are mechanically and biochemically heterogeneous, with areas of soft, normal lung tissue and stiffer regions of mature fibrosis. The modified H5-IgG1 antibodies and fragments thereof of the presently disclosed subject matter can be used to delineate regions of high ECM strain that also present an enhanced αv integrin binding character due to the conformation of the integrin binding domain, perhaps indicative of ongoing fibrosis.

The ability of the modified H5-IgG1 antibody to extract structural information from the ECM can also be demonstrated in a model of retinal angiogenesis, the process by which new blood vessels form by from endothelial sprouting (Patan, 2004). In mouse tissue sections, regions of high modified H5-IgG1:Fn ratio can be found at the extensions of endothelial tip cells, suggesting that Fn is unfolded in these regions. Fn is known to be a mediator of retinal angiogenesis, wherein astrocytes deposit fibronectin prior to differentiation of angioblasts to endothelial cells (Jiang et al., 1994). The results set forth herein suggest that forces from endothelial tip cells unfold Fn, presenting an αvβ3 binding character within the provisional matrix that can influence the formation of new blood vessels.

Described herein in some embodiments is a conformation-sensitive single-chain antibody based on the modified H5-IgG1 of the presently disclosed subject matter to the integrin binding FnIII9-10 domain of Fn and demonstrated its mechano-sensitive binding to Fn in multiple model systems in vitro and ex vivo. While not wishing to be bound by any particular theory of operation, these force-sensitive conformational changes observed in the integrin binding domain of Fn are seen as evidence of the long theorized Fn “integrin-switch” which likely regulates integrin-specific cell responses based on controlling the presentation and accessibility of Fn epitopes in vivo and in engineered contexts. It is also provided herein that the exemplary modified H5-IgG1 antibody specifically detects a force-induced conformational change within a protein. As mechanics of tissues are becoming increasingly implicated in pathogenesis of fibrotic diseases, there is a nascent opportunity to explore targeting the mechanochemical character of ECM as a paradigm for tissue imaging and disease diagnosis.

### I. Abbreviations and Definitions

Certain abbreviations employed in the instant disclosure and/or claims are summarized in Table 1.

TABLE 1
Table of Abbreviations

| | |
|---|---|
| βA | beta alanine |
| ECM | extracellular matrix |
| Fn or FN | fibronectin |
| HFF | human foreskin fibroblast |
| K_{D} | dissociation constant |
| PDMS | polydimethylsiloxane |
| RGD | Arg-Gly-Arg |
| RT | room temperature |
| sc | single chain |
| SPR | surface plasmon resonance |
| TMB | 3,3′,5,5′-tetramethylbenzidine |

Headings are included herein for reference and to aid in locating certain sections. These headings are not intended to limit the scope of the concepts described therein under, and these concepts can have applicability in other sections throughout the entire specification.

In describing and claiming the presently disclosed subject matter, the following terminology will be used in accordance with the definitions set forth below. The terminology used herein is for the purpose of describing particular embodiments only and is not intended to be limiting of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed subject matter and the claims.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The articles “a” and “an” are used herein to refer to one or to more than one (i.e., to at least one) of the grammatical object of the article. By way of example, “an element” means one element or more than one element without limitation unless expressly stated.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In some embodiments, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the term “adjuvant” refers to a substance that elicits an enhanced immune response when used in combination with a specific antigen.

As use herein, the terms “administration of” and or “administering” a composition should be understood to mean providing a composition of the presently disclosed subject matter to a subject in need of treatment.

As used herein, an “agent” is meant to include something being contacted with a cell, tissue, or organ to elicit an effect, such as a drug, a protein, an antibody and/or a fragment or derivative thereof, etc.

The terms “additional therapeutically active compound” or “additional therapeutic agent”, as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which might not be responsive to the primary treatment for the injury, disease or disorder being treated. Diseases and disorders being treated by the additional therapeutically active agent include, for example, cancer, fibrosis, etc. The additional compounds can also be used to treat symptoms associated with the injury, disease, or disorder, including, but not limited to, pain and inflammation.

As used herein, an “agonist” is a composition of matter which, when administered to a mammal such as a human, enhances or extends a biological activity attributable to the level or presence of a target biologically active molecule of interest in the mammal.

An “antagonist” is a composition of matter which when administered to a mammal such as a human, inhibits a biological activity attributable to the level or presence of a biologically active molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in Table 2:

TABLE 2
Amino Acids and Codes Therefor

| | 3-Letter | 1-Letter |
|---|---|---|
| Full Name | Code | Code |
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides (e.g., the antibodies, fragments, and derivatives thereof of the presently disclosed subject matter), and particularly at the carboxy- or amino-terminus, can be modified by methylation, amidation, acetylation or substitution with other chemical groups which can change the peptide's circulating half-life without adversely affecting their activity. Additionally, a disulfide linkage can be present or absent in the peptides of the presently disclosed subject matter.

The term “amino acid” is used interchangeably with “amino acid residue”, and can refer to a free amino acid and to an amino acid residue of a peptide. It will be apparent from the context in which the term is used whether it refers to a free amino acid or a residue of a peptide. Amino acids have the following general structure:

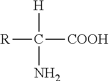

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the antibodies, fragments, and derivatives thereof of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fv, single chain Fv, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fab₁ monomer. The Fab₁ monomer is essentially an Fab with part of the hinge region (see Paul, 1993). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antimicrobial agents” as used herein refers to any naturally-occurring, synthetic, or semi-synthetic compound or composition or mixture thereof, which is safe for human or animal use as practiced in the methods of this presently disclosed subject matter, and is effective in killing or substantially inhibiting the growth of microbes. “Antimicrobial” as used herein, includes antibacterial, antifungal, and antiviral agents.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific or selective binding to their natural ligand or of performing the function of the protein.

The term “biological sample”, as used herein, refers to samples obtained from a subject, including, but not limited to, skin, hair, tissue, blood, plasma, cells, sweat and urine.

The terms “cell” and “cell line”, as used herein, can be used interchangeably. All of these terms also include their progeny, which are any and all subsequent generations. It is understood that all progeny might not be identical due to deliberate or inadvertent mutations.

The terms “cell culture” and “culture”, as used herein, refer to the maintenance of cells in an artificial, in vitro environment. It is to be understood, however, that the term “cell culture” is a generic term and can be used to encompass the cultivation not only of individual cells, but also of tissues, organs, organ systems or whole organisms, for which the terms “tissue culture”, “organ culture”, “organ system culture” or “organotypic culture” can occasionally be used interchangeably with the term “cell culture”.

The phrases “cell culture medium”, “culture medium” (plural “media” in each case) and “medium formulation” refer to a nutritive solution for cultivating cells and can be used interchangeably.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

A “compound”, as used herein, refers to any type of substance or agent that is commonly considered a drug, or a candidate for use as a drug, combinations, and mixtures of the above, as well as polypeptides and antibodies of the presently disclosed subject matter.

As used herein, the term “conservative amino acid substitution” is defined herein as an amino acid exchange within one of the five groups summarized in Table 3.

TABLE 3
Conservative Amino Acid Substitutions

| Group | Characteristics | Amino Acids |
|---|---|---|
| A. | Small aliphatic, nonpolar, or slightly polar residues | Ala, Ser, Thr, Pro, Gly |
| B. | Polar, negatively charged residues and their amides | Asp, Asn, Glu, Gln |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A tissue “normally comprises” a cell if one or more of the cell are present in the tissue in an animal not afflicted with a disease or disorder.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular, and helical domains or properties such as ligand binding, signal transduction, cell penetration and the like. Specific examples of binding domains include, but are not limited to, DNA binding domains, ATP binding domains, and fibronectin's integrin binding domain.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), can be different from when that compound is administered alone. Thus, an effective amount of a combination of compounds refers collectively to the combination as a whole, although the actual amounts of each compound can vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide (e.g., an antibody or a fragment or derivative thereof of the presently disclosed subject matter), can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length. In some embodiments, a fragment of an antibody of the presently disclosed subject matter comprises a paratope.

As used herein, the term “fragment” as applied to a nucleic acid, can ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized. “Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs of Altschul et al., 1990, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit” as used herein means to suppress or block an activity or function such that it is lower relative to a control value. The inhibition can be via direct or indirect mechanisms. In some embodiments, the activity is suppressed or blocked by at least 10%, in some embodiments by at least 25%, and in some embodiments by at least 50% compared to a control value.

The term “inhibitor” as used herein, refers to any compound or agent, such as but not limited to the antibodies, fragments, and derivatives thereof of the presently disclosed subject matter, the application of which results in the inhibition of a process or function of interest, including, but not limited to, expression, levels, and activity. Inhibition can be inferred if there is a reduction in the activity or function of interest.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time. In some embodiments, the antibodies, fragments, and derivatives thereof of the presently disclosed subject matter are protein inhibitors.

As used herein “injecting or applying” includes administration of a composition of the presently disclosed subject matter by any number of routes and means including, but not limited to, intravitreal, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

The term “injury” refers to any physical damage to the body caused by violence, accident, trauma, or fracture, etc., as well as damage by surgery.

As used herein, an “instructional material” includes a publication, a recording, a diagram, or any other medium of expression which can be used to communicate the usefulness of the peptides (e.g., antibodies, fragments and derivatives thereof) of the presently disclosed subject matter in the kit for effecting alleviation of the various diseases or disorders recited herein. Optionally, or alternately, the instructional material can describe one or more methods of alleviating the diseases or disorders in a cell or a tissue of a mammal. The instructional material of the kit of the presently disclosed subject matter can, for example, be affixed to a container which contains an identified antibody, fragment, and/or derivative thereof of the presently disclosed subject matter or be shipped together with a container which contains the identified antibody, fragment, and/or derivative thereof. Alternatively, the instructional material can be shipped separately from the container with the intention that the instructional material and the antibody, fragment, and/or derivative thereof of the presently disclosed subject matter be used cooperatively by the recipient.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

As used herein, a “ligand” is a molecule that specifically or selectively binds to a target molecule. A ligand (e.g., an antibody) “specifically binds to”, “is specifically immunoreactive with”, “having a selective binding activity”, “selectively binds to” or “is selectively immunoreactive with” a molecule when the ligand functions in a binding reaction which is determinative of the presence of the molecule in a sample of heterogeneous molecules. Thus, under designated assay (e.g., immunoassay) conditions, the ligand binds preferentially to a particular molecule and does not bind to a significant extent to other molecules present in the sample. For example, an antibody specifically or selectively binds under immunoassay conditions to an antigen bearing an epitope against which the antibody was raised. A variety of immunoassay formats can be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a molecule that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e. analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides. However, in some embodiments the term “peptide” refers to an antibody, or a fragment or derivative thereof, of the presently disclosed subject matter. As such, in some embodiments the term “peptide” refers to an intact antibody.

The term “per application” as used herein refers to administration of any molecule (e.g., the antibodies, fragments, and derivatives thereof of the presently disclosed subject matter) to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the U.S. Federal government or listed in the U.S. Pharmacopeia for use in animals, including humans.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide can be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but can be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

The term “propagate” means to reproduce or to generate.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus. In some embodiments of the presently disclosed subject matter, a protein is an antibody or a fragment or derivative thereof.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” molecule or compound as used herein refers to a molecule or compound that is greater than 90% pure. Representative purification techniques are disclosed herein for antibodies and fragments and derivatives thereof.

“Recombinant polynucleotide” refers to a polynucleotide having sequences that are not naturally joined together. An amplified or assembled recombinant polynucleotide can be included in a suitable vector, and the vector can be used to transform a suitable host cell.

A recombinant polynucleotide can serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” when used in reference to a substrate forming a linkage with a molecule, relates to a solvent insoluble substrate that is capable of forming linkages (in some embodiments covalent bonds) with various molecules. The support can be either biological in nature, such as, without limitation, a cell or bacteriophage particle, or synthetic, such as, without limitation, an acrylamide derivative, agarose, cellulose, nylon, silica, or magnetized particles.

By the term “solid support suitable for maintaining cells in a tissue culture environment” is meant any surface such as a tissue culture dish or plate, or even a cover, where medium containing cells can be added, and that support can be placed into a suitable environment such as a tissue culture incubator for maintaining or growing the cells. This should of course be a solid support that is either sterile or capable of being sterilized. The support does not need to be one suitable for cell attachment.

The term “solid support is a low adherence, ultralow adherence, or non-adherence support for cell culture purposes” refers to a vehicle such as a bacteriological plate or a tissue culture dish or plate which has not been treated or prepared to enhance the ability of mammalian cells to adhere to the surface. It could include, for example, a dish where a layer of agar has been added to prevent cells from attaching. It is known to those of ordinary skill in the art that bacteriological plates are not treated to enhance attachment of mammalian cells because bacteriological plates are generally used with agar, where bacteria are suspended in the agar and grow in the agar.

The term “standard”, as used herein, refers to something used for comparison. For example, a standard can be a known standard agent or molecule which is administered or added to a control sample and used for comparing results when measuring said molecule in a test sample. Standard can also refer to an “internal standard”, such as an agent or molecule which is added at known amounts to a sample and is useful in determining such things as purification or recovery rates when a sample is processed or subjected to purification or extraction procedures before a marker of interest is measured.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, more in some embodiments by at least 25%, and in some embodiments by at least 50%.

The term “stimulator” as used herein, refers to any composition, molecule or agent, the application of which results in the stimulation of a process or function of interest, including, but not limited to, wound healing, angiogenesis, bone healing, osteoblast production and function, and osteoclast production, differentiation, and activity.

A “subject” of diagnosis or treatment is an animal, including a human. It also includes pets and livestock.

As used herein, a “subject in need thereof” is a patient, animal, mammal, or human, who will benefit from the method of this presently disclosed subject matter.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes an amino acid sequence having substantially the same structure and function as an amino acid sequence encoded by the reference nucleic acid sequence; e.g., where only changes in amino acids not significantly affecting the function of the amino acid sequence occur. In some embodiments, the substantially identical nucleic acid sequence encodes the same amino acid sequence encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2X standard saline citrate (SSC), 0.1% SDS at 50° C.; in some embodiments in 7% (SDS), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and more in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a molecule, e.g., a protein or polypeptide which has been separated from components which naturally accompany it. Typically, a molecule is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the molecule of interest. Purity can be measured by any appropriate method, e.g., in the case of polypeptides by column chromatography, gel electrophoresis, or HPLC analysis. A molecule, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a molecule is that amount of molecule which is sufficient to provide a beneficial effect to the subject to which the molecule is administered.

The use of the phrase “tissue culture dish or plate” refers to any type of vessel which can be used to plate cells for growth or differentiation.

The term “thermal injury” is used interchangeably with “thermal burn” herein.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or molecules by topical application of the drug or molecule thereto. “Transdermal” is used interchangeably with “percutaneous”.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat”, as used herein, means reducing the frequency with which symptoms are experienced by a patient or subject or administering an agent or molecule to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic molecules, plasmids, and viruses. Thus, the term “vector” includes an autonomously replicating plasmid or a virus. The term should also be construed to include non-plasmid and non-viral molecules which facilitate transfer or delivery of nucleic acid to cells, such as, for example, polylysine molecules, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

As used herein, the term “wound” relates to a physical tear, break, or rupture to a tissue or cell layer. A wound can occur by any physical insult, including a surgical procedure or as a result of a disease, disorder condition.

Methods useful for the practice of the presently disclosed subject matter which are not described herein are also known in the art. Useful methods include those described in PCT International Patent Application Publication Nos. WO 2007/019107; WO 2007/030652; WO 2007/089798; WO 2008/060374, the methods of which are hereby incorporated by reference.

### II. Exemplary Embodiments

In some embodiments, the presently disclosed subject matter provides compositions and methods useful for targeting of a mechanically exposed cryptic site within fibronectin's integrin binding domain. Thus, in some embodiments, the presently disclosed subject matter provides methods for targeting conformational states of fibronectin (FN) in samples, optionally biological samples isolated from or present within a subject. In some embodiments, the methods comprise contacting a sample with a composition having a selective binding activity for a conformational state of FN comprising FnIII9-4G-10 (4G), whereby the conformational state is targeted. In some embodiments, the sample comprises or is suspected to comprise a tissue undergoing tissue repair, a tissue that is diseased, a tissue that suffers from a disorder, or any combination thereof.

The presently disclosed subject matter provides for the detection of an integrin-binding mechanoswitch within fibronectin (Fn) during tissue formation and fibrosis. The disclosed results demonstrate the in vivo existence and activation of the long theorized Fn conformational switch within the integrin binding domain and suggest its influence in skewing integrin specificity in both developmental processes, as well as in pathological tissue fibrosis. Antibodies such as the presently disclosed modified H5-IgG1 antibody thus represent an attractive approach to detecting and targeting key developmental and disease processes.

Binding kinetics and activity of a modified H5 antibody are disclosed herein. The modified H5 antibody recognizes FnIII9, also referred to herein as FnIII9-4G-10 (4G), and it selectively inhibits αvβ3 binding to Fn-absorbed surfaces.

Particularly, the modified H5 antibody of the presently disclosed subject matter has been generated by combining the variable regions of the H5 antibody with the universal constant regions of the IgG1 subclass. Nucleic acid sequences encoding the heavy chain variable region of the H5 antibody were combined with sequences encoding IgG1 subclass constant regions of IgG1 including CH1, CH2, and CH3. Similarly, nucleotide sequences encoding the light chain variable region of the H5 antibody were combined with nucleic acid sequences encoding the constant light (CL) chain-kappa. To express the modified H5 antibody in full length IgG1 format, a donor construct encoding an H5-light chain variable region and a H5-heavy chain variable region was stably integrated into the immunoglobulin loci (IgG1) of the genome of the hybridoma cells by Cas9 targeting. The culture supernatants of the cells expressing the modified H5-IgG1 antibody were analyzed for the presence of the IgG, quantified, and used in an ELISA binding assay described in EXAMPLE 1. For the routine large-scale production of H5-IgG1 in mammalian cells, H5-heavy chain variable region and a H5-light chain variable region sequence can be cloned separately into two independent IgG1 expression vector back bones by Gibson Assembly® Cloning, NEBUILDER® HiFi DNA Assembly (New England Biolabs, Ipswich. Massachusetts, United States of America), IN-FUSION® Snap Assembly (Takara Bio USA, Inc., Mountain View, California, United States of America), or any other strategy. These plasmids encode the constant regions of human IgG1 heavy chain and kappa light chains respectively, providing a framework for the insertion of variable regions of choice. For the transient expression of H5-IgG1, the plasmids containing the H5-heavy chain and H5-light chain are co-transfected to the mammalian suspension cells of choice such as 293-6E or Chinese hamster ovary (CHO) cells. The culture supernatants are collected between 48-96 hours post transfection and used for the isolation of the full length H5-IgG1 by Protein L or Protein G chromatography.

The modified H5-IgG1 antibody and antigen-binding fragments thereof were characterized by a much higher affinity for the FN fragments showing at least 50-fold higher binding when compared with equimolar concentration of the parental H5 antibodies. Further, the modified H5-IgG1 antibody showed higher affinity for the FN-4G-10 (i.e., diseased) form as compared to the FN-9*-10 (healthy) form.

As such, the presently disclosed subject matter provides compositions and methods useful for detecting distinct conformational states of Fn. In some embodiments, binding of modified H5-IgG1 antibody and antigen-binding fragments thereof to Fn can detect distinct conformational states of Fn, such as FnIII9-4G-10 (4G).

The presently disclosed subject matter provides in some embodiments compositions and methods useful for detecting distinct conformational states of Fn and the binding ratios are useful for distinguishing normal tissue from tissue that is diseased or suffers from a disorder. In some embodiments, the severity of fibrosis in a tissue can be determined. In some embodiments, the presently disclosed subject matter is useful for detecting transient, force-induced conformational change in Fn. The compositions and methods are useful for targeting early molecular signatures of cell contractile forces during tissue repair. The compositions and 30 methods are useful for targeting early molecular signatures of cell contractile forces in diseases and disorders.

In some embodiments, the presently disclosed subject matter provides compositions and methods useful for detecting and comparing pathologic ECMs. In some embodiments, the presently disclosed subject matter provides compositions and methods useful for detecting and distinguishing fibrotic ECMs.

In some embodiments, the presently disclosed subject matter provides compositions and methods useful for delineating regions of high ECM strain. In some embodiments, the high ECM strain is associated with enhanced αv integrin binding character due to the conformation of the integrin binding domain, perhaps indicative of ongoing fibrosis.

In some embodiments, an antibody of the presently disclosed subject matter is useful for extracting structural information from the ECM.

In some embodiments, the compositions and methods are useful for determining antibody: Fn ratios, which in turn can be used for diagnosing or distinguishing normal from diseased tissue and for determining the type of treatment to be administered when the subject has been diagnosed with a disease or disorder.

The presently disclosed subject matter provides other antibodies and biologically active fragments and homologs thereof as well as methods for preparing and testing new antibodies for the properties disclosed herein.

In some embodiments, an antibody or biologically active fragment or homolog thereof is useful for treating a disease or disorder associated with the fibronectin-interaction signaling pathway disclosed herein. In some embodiments, the pathway is regulated by a mechanoswitch.

In some embodiments, the presently disclosed subject matter uses a biologically active antibody or biologically active fragment or homolog thereof. In some embodiments, the isolated polypeptide comprises a mammalian molecule at least about 30% homologous to a polypeptide having the amino acid sequence of at least one of the sequences disclosed herein. In some embodiments, the isolated polypeptide is at least about 35% homologous, more in some embodiments, about 40% homologous, more in some embodiments, about 45% homologous, in some embodiments, about 50% homologous, more in some embodiments, about 55% homologous, in some embodiments, about 60% homologous, more in some embodiments, about 65% homologous, in some embodiments, more in some embodiments, about 70% homologous, more in some embodiments, about 75% homologous, in some embodiments, about 80% homologous, more in some embodiments, about 85% homologous, more in some embodiments, about 90% homologous, in some embodiments, about 95% homologous, more in some embodiments, about 96% homologous, more in some embodiments, about 97% homologous, more in some embodiments, about 98% homologous, and most in some embodiments, about 99% homologous to at least one of the peptide sequences disclosed herein.

The presently disclosed subject matter further encompasses modification of the antibodies and fragments thereof disclosed herein, including amino acid deletions, additions, and substitutions, particularly conservative substitutions. The presently disclosed subject matter also encompasses modifications to increase in vivo half-life and decrease degradation in vivo. Substitutions, additions, and deletions can include, for example, 1, 2, 3, 4, 5, 6, 7, 8, 9, 10, 11, 12, 13, 14, 15, 16, 17, 18, 19, 20, 21, 22, 23, 24, and 25 changes as long as the activity disclosed herein remains substantially the same.

The presently disclosed subject matter includes an isolated nucleic acid comprising a nucleic acid sequence encoding an antibody of the presently disclosed subject matter, or a fragment or homolog thereof. In some embodiments, the nucleic acid sequence encodes a peptide comprising an antibody sequence of the presently disclosed subject matter, or a biologically active fragment of homolog thereof.

In some embodiments, a homolog of a peptide (antibody or fragment) of the presently disclosed subject matter is one with one or more amino acid substitutions, deletions, or additions, and with the sequence identities described herein. In some embodiments, the substitution, deletion, or addition is conservative. In some embodiments, a serine or an alanine is substituted for a cysteine residue in a peptide of the presently disclosed subject matter.

In some embodiments, the subject is a mammal. In some embodiments, the mammal is a human.

The presently disclosed subject matter encompasses the use of purified isolated, recombinant, and synthetic peptides.

The presently disclosed subject matter further encompasses the use of drugs or other molecules that can target the exposed cryptic site disclosed herein and can, for example, recognize the conformational changes disclosed herein and have the same activity disclosed herein.

Thus, the presently disclosed subject matter provides in some embodiments methods for detecting conformational states of fibronectin (FN) in samples. In some embodiments, the methods comprise contacting a sample with a composition having a selective binding activity for a conformational state of FN comprising FnIII9-4G-10 (4G); and detecting the binding of the composition, whereby the conformational state of FN is detected. In some embodiments, the sample comprises or is suspected to comprise a tissue undergoing tissue repair, a tissue that is diseased, a tissue that suffers from a disorder, or a combination thereof. In some embodiments, the sample comprises or is suspected to comprise a pathologic extracellular matrix (ECM). In some embodiments, the sample comprises or is suspected to comprise a fibrotic ECM.

In some embodiments, detecting the binding of the composition comprises distinguishing normal from diseased tissue. In some embodiments, detecting the binding of the composition comprises determining severity of fibrosis in the sample, such as by using a binding ratio of compositon to FN. In some embodiments, detecting the binding of the composition comprises detecting a transient, force-induced conformational change in FN. In some embodiments, detecting the binding of the composition comprises extracting structural information for an ECM in the sample. In some embodiments, extracting structural information for an ECM in the sample comprises delineating regions of high ECM strain. In some embodiments, the high ECM strain is associated with enhanced αv integrin binding character.

In some embodiments, the methods further comprise determining a type of treatment to be administered to the subject based on the detecting of the binding of the composition.

The presently disclosed subject matter also provides in some embodiments methods for treating diseases and/or disorders in subjects. In some embodiments, the methods comprise administering to a subject in need there of a therapeutically effective amount of a composition having a selective binding activity for a conformational state of FN comprising FnIII9-4G-10 (4G), whereby treatment is accomplished. In some embodiments, the disease and/or disorder has a characteristic selected from the group consisting of a tissue undergoing tissue repair, a tissue that is diseased, a tissue that suffers from a disorder, and any combination thereof. In some embodiments, the characteristic is a pathologic extracellular matrix (ECM). In some embodiments, the characteristic is a fibrotic ECM.

In some embodiments of the presently disclosed methods, the composition having a selective binding activity for a conformational state of FN comprising FnIII9-4G-10 (4G) is an isolated and purified antibody comprising an amino acid sequence selected from the group consisting of SEQ ID NOs: 2, 4, 6, 14, 16, or 18, a fragment thereof, or an antibody have a sequence approximately 95% identical to a sequence of SEQ ID NOs: 2, 4, 6, 14, 16, or 18, or a fragment thereof. In some embodiments, the amino acid sequence comprises at least one modification selected from the group consisting of an amino acid deletion, an amino acid addition, an amino acid substitution, and combinations thereof. In some embodiments, the isolated antibody is present in multiple copies (i.e., is multimerized), with each copy linked by a linker. Multiple copies can include in some embodiments two copies, in some embodiments three copies, in some embodiments four copies, in some embodiments five copies, and in some embodiments more than five copies. In some embodiments, the individual members of the multimeric composition are identical to each other, and in some embodiments one or more of the individual members of the multimeric composition is different from at least one other individual member of the multimeric composition.

The presently disclosed subject matter also provides in some embodiments methods for screening for molecules having selective binding activities for conformational states of FN comprising FnIII9-4G-10 (4G). In some embodiments, the methods comprise providing a sample comprising a conformational state of FN comprising FnIII9-4G-10 (4G); contacting the sample with a candidate molecule; and detecting binding of the candidate molecule to the sample. In some embodiments, the candidate molecule is a member of a library of molecules. In some embodiments, the candidate compound is a small molecule or an antibody. In some embodiments, the conformational state of FN is a force-induced conformational change in Fn.

The presently disclosed subject matter also provides in some embodiments compounds identified by the presently disclosed methods.

The presently disclosed subject matter also provides in some embodiments methods for treating diseases and/or disorder in subjects comprising administering to a subject in need thereof a therapeutically effective amount of a composition comprising an antibody or fragment thereof in accordance with the presently disclosed subject matter, whereby treatment is accomplished.

The presently disclosed subject matter also provides in some embodiments methods for ameliorating at least one symptom of consequence of a disease or disorder associated with abnormal expression of a force-induced conformational state of FN comprising FnIII9-4G-10 (4G) in a subject. In some embodiments, the methods comprise administering to a subject in need thereof a therapeutically effective amount of a composition comprising an antibody or fragment thereof in accordance with the presently disclosed subject matter, wherein at least one symptom of consequence of a disease or disorder associated with abnormal expression of a force-induced conformational state of FN comprising FnIII9-4G-10 (4G) is ameliorated.

In some embodiments of the therapeutic methods, the disease or disorder is associated with a tissue undergoing tissue repair, a tissue that is diseased, a tissue that suffers from a disorder, or any combination thereof. In some embodiments, the disease or disorder is associated with a pathologic extracellular matrix (ECM). In some embodiments, the disease or disorder is associated with a fibrotic ECM.

### III. Exemplary Sequences of the Presently Disclosed Subject Matter

The presently disclosed subject matter provides for the use of various antibodies with the activity described herein as well as biologically active fragments and homologs thereof.

In some embodiments, the presently disclosed subject matter provides isolated and purified antibodies that comprise an amino acid sequence selected from the group consisting of SEQ ID NOs: 2, 4, 6, 14, 16, or 18, a fragment thereof, an antibody having an amino acid sequence that is approximately 95% identical to the sequence of any one of SEQ ID NOs: 2, 4, 6, 14, 16, or 18, a fragment thereof, and and substantially homologous amino acid sequences of any of the foregoing sequences. In some embodiments, the amino acid sequence comprises at least one modification selected from the group consisting of an amino acid deletion, an amino acid addition, an amino acid substitution, and combinations thereof. In some embodiments, the antibody or fragment thereof is humanized.

In some embodiments, the isolated and purified antibody, or fragment or homolog thereof, comprises a heavy chain CDR1 of sequence SYAMS (SEQ ID NO: 8), a heavy chain CDR2 of sequence DIYDGGGTNYADSVKG (SEQ ID NO: 10), a heavy chain CDR3 of sequence TADNFDY (SEQ ID NO: 12), a light chain CDR1 of sequence RASQSISSYLN (SEQ ID NO: 20), a light chain CDR2 of sequence AASTLQS (SEQ ID NO: 22), and a light chain CDR3 of sequence QQANSAPTT (SEQ ID NO: 24).

In some embodiments, the isolated and purified antibody, or fragment or homolog thereof, comprises a heavy chain framework region 1 comprising EVQLLESGGGLVQPGGSLRLSCAAS (SEQ ID NO: 40), a heavy chain framework region 2 comprising WVRQAPGKGLEWV (SEQ ID NO: 41), a heavy chain framework region 3 comprising RFTTSRDNSKNTLYLQMNSLRAEDTAVYYC (SEQ ID NO: 42), and a heavy chain framework region 4 comprising WGQGTLVTVSS (SEQ ID NO: 43); and/or the isolated and purified antibody further comprises a light chain framework region 1 comprising DIQMTQSPSSLSASVGDRVTITC (SEQ ID NO: 44), a light chain framework region 2 comprising WYQQKPGKAPKLLIY (SEQ ID NO: 45), a light chain framework region 3 comprising GVPSRFSGSGSGTDFTLTISSLQPEDFATYYC (SEQ ID NO: 46), and a light chain framework region 4 comprising FGQGTKVEIK (SEQ ID NO: 47).

In some embodiments, the isolated and purified antibody, or fragment or homolog thereof, comprises a modification at its N-terminus, its C-terminus, or both. In some embodiments, the modification comprises addition of a peptide tag, a SARAH domain, or a combination thereof. In some embodiments, the tag comprises a his tag, a myc tag, a VSV tag, an HA tag, a SortaseA tag, a PelB sequence, or any combination of one or more thereof. In some embodiments, the His tag comprises, consists essentially of, or consists of the amino acid sequence HHHHHH (SEQ ID NO: 35). In some embodiments, the myc tag comprises, consists essentially of, or consists of the amino acid sequence EQKLISEEDL (SEQ ID NO: 33). In some embodiments, the VSV tag comprises, consists essentially of, or consists of the amino acid sequence YTDIEMNRLGK (SEQ ID NO: 34). In some embodiments, the HA tag comprises, consists essentially of, or consists of the amino acid sequence YPYDVPDYA (SEQ ID NO: 36). In some embodiments, the SortaseA tag comprises, consists essentially of, or consists of the amino acid sequence LPXTG (SEQ ID NO: 48), wherein the X at amino acid 3 of SEQ ID NO: 48 can be any amino acid. In some embodiments, the PelB sequence comprises, consists essentially of, or consists of the amino acid sequence MKYLLPTAEAGLLLLLAAPQIA (SEQ ID NO: 49). In some embodiments, an isolated and purified antibody, or fragment or homolog thereof of the presently disclosed subject matter comprises any combination of one or more of a His tag, a myc tag, a VSV tag, HA tag, a SortaseA tag, and a PelB sequence, including any combination of one or more of these sequences or even more than one of one or more of the listed sequences. In some embodiments, the SARAH domain comprises a sequence selected from the group consisting of SEQ ID NOs: 28-32.

The presently disclosed subject matter also provides in some embodiments isolated and purified nucleic acid sequences encoding the antibodies and fragments disclosed herein, and substantially homologous nucleic acid sequences thereto.

The presently disclosed subject matter also provides in some embodiments recombinant nucleic acids and substantially homologous nucleic acid sequences thereto. In some embodiments, the recombinant nucleic acids comprise a first nucleic acid segment encoding a V_{H} segment comprising a first amino acid sequence comprising SEQ ID NO: 4, a V_{L} segment comprising a second amino acid sequence comprising SEQ ID NO: 16, or a combination thereof, wherein the first and second segments are optionally present in a same reading frame. In some embodiments, the recombinant nucleic acids further comprise a third nucleic acid segment encoding a linker peptide coupling together the first and second segments in frame. In some embodiments, the recombinant nucleic acids further comprise one or more additional nucleic acid segments that encode one or more subsequences of an intact antibody, such that the recombinant nucleic acid encodes a recombinant intact antibody.

In some embodiments, the presently disclosed antibodies, fragments, and homologs thereof can comprise a tag sequence, linker sequence, spacer sequence and/or other additional sequence that can be used in to facilitate expression, stability, purification, isolation, or other desired feature or aspect. Multiple copies of such sequences can be employed. Such sequences can be added to the N-terminus, the C-terminus, or both of an antibody, fragment, or homolog thereof of the presently disclosed subject matter. Representative such sequences include SARAH sequences. SEQ ID NOs: 28-32 are the amino acid sequences of exemplary SARAH domains that can be added to the N-terminus, the C-terminus, or both an antibody, fragment, or homolog thereof of the presently disclosed subject matter. Representative such sequences also include tags sequences such as myc, VSV, His, HA, SortaseA, and PelB tags. By way of particular example and not limitation, SEQ ID NOs: 33-38 are the amino acid sequences of exemplary tags that can be added to the N-terminus, the C-terminus, or both of that can be added to the N-terminus, the C-terminus, or both an antibody, fragment, or homolog thereof of the presently disclosed subject matter. EQKLISEEDL (SEQ ID NO: 33) is an exemplary myc tag, YTDIEMNRLGK (SEQ ID NO: 34) is an exemplary VSV tag, HHHHHH (SEQ ID NO: 35) is an exemplary His tag, YPYDVPDYA (SEQ ID NO: 36) is an exemplary HA tag, LPTEGG (SEQ ID NO: 37) is an exemplary Sortase A tag, and MKYLLPTAAAGLLLLAAQPAMA (SEQ ID NO: 38) is an exemplary PelB tag.

In some embodiments, an antibody or fragment thereof in accordance with the presently disclosed subject matter is modified by a SARAH domain. Representative approaches for implementing SARAH domains are described in European Patent Application No. 17868682.0; PCT International Patent Application Publication No. WO 2018/088403; U.S. patent application Ser. No. 16/345,639; and in Arimori et al., 2017, each of are herein wherein incorporated by reference in their entireties. In some embodiments, an N-terminus of a SARAH domain is linked to a C-terminus of a heavy chain domain (V_{H} region) and/or to a C-terminus of a light chain domain (V_{L} region) of an antibody or fragment thereof in accordance with the presently disclosed subject matter. A SARAH domain is a domain (peptide) comprising a short helix (hl) on the N-terminal side and a long helix (h2) on the C-terminal side, which in some embodiments comprises usually 42 to 54, optionally 43 to 49, further optionally 47 to 49, and yet further optionally 49 amino acid residues and has properties of forming antiparallel coiled coils between h2 with another SARAH domain. It should be noted that hl can comprise 5 to 7 amino acid residues, and h2 can comprise 38 to 42 pieces of amino acid residues.

The SARAH domain used here can be any SARAH domain as would be suitable to one of ordinary skill in the art upon a review on the instant disclosure. By way of further example and not limitation, in a case where two SARAH domains form an antiparallel coiled coil between h2, the distance between both N-termini of two SARAH domains (two hl) is optionally about 35A to 45A, further optionally about 39A to 41A, and further optionally about 40A. Particular examples of such SARAH domains include the SARAH domain of the human mammalian sterile 20-like kinase 1 polypeptide (hMST1; DYEFLKSWTVEDLQ KRLLALDPMMEQEIEEIRQKYQSKRQPILDAIEAK, SEQ ID NO: 28; corresponding to amino acids 432-480 of Accession No. NP_006273.1 of the GENBANK® biosequence database), the SARAH domain of the human mammalian sterile 20-like kinase 2 polypeptide (hMST2; DFDFLKNLSLEELQMRLKALDPMMEREIEELRQRYTAKRQPILDAMDAK, SEQ ID NO: 29; corresponding to amino acids 325-373 of Accession No. NP_001243242.1 of the GENBANK® biosequence database), the SARAH domain of the human ras association domain-containing protein in 0 polypeptide (hRAF5; GEVEWDAFSIPE LQNFLTILEKEEQDKIQQVQKKYDKFRQKLEEALRES, SEQ ID NO: 30; corresponding to amino acids 212-260 of Accession No. NP_872606.1 of the GENBANK® biosequence database), the SARAH domain of the human ras association domain-containing protein 1 isoform B polypeptide (hRAF1; GEVNWDAFSMPELHNFLRILQR EEEEHLRQILQKYSYSRQKIQEALHAS, SEQ ID NO: 31; which shows 47/49 amino acid identity to amino acids 138-186 of Accession No. NP_001193886.1 of the GENBANK® biosequence database), the SARAH domain of the human protein salvador homolog 1 polypeptide (hSAVI; HILKWELFQLADLDTYQGMLKLLFMKELEQIVKMYEAYRQ ALLTELENR, SEQ ID NO: 32; corresponding to amino acids 320-368 of Accession No. NP_068590.1 of the GENBANK® biosequence database), and further include those having a sequence homology of in some embodiments 85% or more, in some embodiments 90% or more, and in some embodiments 95% or more with one of the foregoing SARAH domains. With respect to the foregoing representative SARAH domains, a general design approach can involve heavy and light chains of a given antibody being individually fused with a 49-residue SARAH domain via a two-residue (Gly-Ser) linker. In some embodiments with respect to the foregoing representative SARAH domains, two Cys residues (38 and 49) in the hRAF1 SARAH domain were substituted with Ser to avoid undesired disulfide bond formation. In some embodiments, residues 24 and 35 are mutated to Cys to form an asymmetric inter-chain disulfide bond based on the homodimeric hMST1 structure.

One of ordinary skill in the art will appreciate that based on the sequences of the components of the antibodies disclosed herein they can be modified independently of one another with conservative amino acid changes, including, insertions, deletions, and substitutions, and that the valency could be altered as well. Amino acid changes (fragments and homologs) can be made independently in an antibody as well when they are being used in combination therapy.

In some embodiments, a protein or peptide of the presently disclosed subject matter, or a combination thereof, can be administered by a route selected from, including, but not limited to, intravenously, intrathecally, locally, intramuscularly, topically, orally, intra-arterially, parenterally, etc. Administration can be more than once. One of ordinary skill in the art can determine how often to administer the molecule, the dose to be used, and what combination of other agents it can be administered with such as therapeutic agents and/or other drugs or molecules such as antimicrobial agents, anti-inflammatory agents, etc. One of ordinary skill in the will be able to determine when or if to use an additional agent and the route of administration.

In some embodiments, the present proteins or polypeptides are administered by injection. The parenteral route for administration of the polypeptide is in accordance with known methods, e.g., injection or infusion by intravenous, intraperitoneal, intramuscular, intra-arterial, subcutaneous, or intralesional routes. The protein or polypeptide can be administered continuously by infusion or by bolus injection. A typical composition for intravenous infusion could be made up to contain 10 to 50 ml of sterile 0.9% NaCl or 5% glucose optionally supplemented with a 20% albumin solution and between 10 ug and 50 mg, in some embodiments between 50 ug and 10 mg, of the polypeptide. A typical pharmaceutical composition for intramuscular injection would be made up to contain, for example, 1-10 ml of sterile buffered water and between 10 ug and 50 mg, in some embodiments between 50 ug and 10 mg, of the polypeptide of the presently disclosed subject matter. Methods for preparing parenterally administrable compositions are well known in the art and described in more detail in various sources, including, for example, Genaro 1985, which is incorporated herein by reference in its entirety for all purposes.

When used in vivo for therapy, the antibodies of the subject presently disclosed subject matter are administered to the subject in therapeutically effective amounts (i.e., amounts that have desired therapeutic effect). They will normally be administered parenterally. The dose and dosage regimen will depend upon the degree of the disease or disorder, the characteristics of the particular antibody or immunotoxin used, e.g., its therapeutic index, the patient, and the patient's history. Advantageously the antibody or fragment thereof is administered continuously over a period of 1-2 weeks. Optionally, the administration is made during the course of adjunct therapy such as antimicrobial treatment, or administration of tumor necrosis factor, interferon, or other cytoprotective or immunomodulatory agent.

For parenteral administration, the antibodies and fragments thereof can be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

The antibody compositions used are formulated and dosages established in a fashion consistent with good medical practice taking into account the condition or disorder to be treated, the condition of the individual patient, the site of delivery of the composition, the method of administration, and other factors known to practitioners. The antibody compositions are prepared for administration according to the description of preparation of polypeptides for administration, infra.

The hybrid antibodies and hybrid antibody fragments include in some embodiments complete antibody molecules having full length heavy and light chains, or any fragment thereof, including but not limited to fragments that bind to antigens (e.g., comprise a paratope). Chimeric antibodies which have variable regions as described herein and constant regions from various species are also suitable. See for example, U.S. Patent Application Publication No. 2003/0022244.

The peptides of the presently disclosed subject matter can be readily prepared by standard, well-established techniques, such as solid-phase peptide synthesis (SPPS) as described by Stewart et al., 1984; Bodanszky & Bodanszky, 1984. At the outset, a suitably protected amino acid residue is attached through its carboxyl group to a derivatized, insoluble polymeric support, such as cross-linked polystyrene or polyamide resin.

“Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis can be conducted using high resolution mass spectrometry to determine the molecular weight of the peptide. Alternatively, or additionally, the amino acid content of the peptide can be confirmed by hydrolyzing the peptide in aqueous acid, and separating, identifying, and quantifying the components of the mixture using HPLC, or an amino acid analyzer. Protein sequenators, which sequentially degrade the peptide and identify the amino acids in order, can also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures can be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein can be purified by following known procedures for protein purification, wherein an immunological, enzymatic, or other assay is used to monitor purification at each stage in the procedure. Protein purification methods are well known in the art, and are described, for example in Deutscher et al. 1990.

### Peptide Modification and Preparation

Methods for producing, modifying, and purified peptides are known. It will be appreciated that the proteins and peptides of the presently disclosed subject matter can incorporate amino acid residues which are modified without affecting activity. For example, the termini can be derivatized to include blocking groups, i.e. chemical substituents suitable to protect and/or stabilize the N- and C-termini from “undesirable degradation”, a term meant to encompass any type of enzymatic, chemical or biochemical breakdown of the molecule at its termini which is likely to affect the function of the molecule, i.e. sequential degradation of the molecule at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

The presently disclosed subject matter also provides for analogs of proteins. Analogs can differ from naturally occurring proteins or peptides by conservative amino acid sequence differences or by modifications which do not affect sequence, or by both. For example, conservative amino acid changes can be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the presently disclosed subject matter are not limited to products of any of the specific exemplary processes listed herein.

The presently disclosed subject matter includes the use of beta-alanine (also referred to as β-alanine, β-Ala, bA, and βA), having the structure:

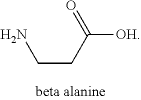

Sequences are provided herein which use the symbol “βA”, but in the Sequence Listing submitted herewith “βA” is provided as “Xaa” and reference in the text of the Sequence Listing indicates that Xaa is beta alanine.

It will be appreciated, of course, that the peptides or antibodies, derivatives, or fragments thereof can incorporate amino acid residues which are modified without affecting activity. For example, the termini can be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH2), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide can include one or more D-amino acid resides, or can comprise amino acids which are all in the D-form. Retro-inverso forms of peptides in accordance with the presently disclosed subject matter are also contemplated, for example, inverted peptides in which all amino acids are substituted with D-amino acid forms.

Substantially pure protein obtained as described herein can be purified by following known procedures for protein purification, wherein an immunological, enzymatic, or other assay is used to monitor purification at each stage in the procedure. Protein purification methods are well known in the art, and are described, for example in Deutscher et al., 1990.

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability, and/or other biological, physical, and chemical properties.

### Amino Acid Substitutions

In certain embodiments, the disclosed methods and compositions can involve preparing peptides with one or more substituted amino acid residues.

In various embodiments, the structural, physical, and/or therapeutic characteristics of peptide sequences can be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide can include one or more D-amino acid resides, or can comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3-or 4-chlorophenylalanine, 2-,3- or 4-methylphenylalanine, 2-,3- or 4-methoxyphenylalanine, 5-amino-, 5-chloro-, 5-methyl-or 5-methoxytryptophan, 2′-, 3′-, or 4′-amino-, 2′-, 3′-, or 4′-chloro-, 2,3, or 4-biphenylalanine, 2′,-3′, - or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids can be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution can also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred. 20) Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine, and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr; Pro (P) Ala; Ser (S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. See e.g., the PROWL Rockefeller University website. For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, Mclachlan matrix, Doolittle matrix, Henikoff matrix, Miyata matrix, Fitch matrix, Jones matrix, Rao matrix, Levin matrix and Risler matrix (see e.g., the PROWL Rockefeller University website)

In determining amino acid substitutions, one can also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### Antibody Formats and Preparation Thereof

Antibodies directed against proteins, polypeptides, or peptide fragments thereof of the presently disclosed subject matter can be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants can be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is a F(ab′)₂, F(ab)₂, Fab′, or Fab fragment.

For the preparation of monoclonal antibodies, any technique which provides for the production of antibody molecules by continuous cell lines in culture can be utilized. For example, the hybridoma technique originally developed by Kohler & Milstein, the trioma technique, the human B-cell hybridoma technique (Kozbor & Roder, 1983), and the EBV-hybridoma technique (Cole et al., 1985) can be employed to produce human monoclonal antibodies. In some embodiments, monoclonal antibodies are produced in germ-free animals.

In accordance with the presently disclosed subject matter, human antibodies can be used and obtained by utilizing human hybridomas (Cote et al., 1983) or by transforming human B cells with EBV virus in vitro (Cole et al., 1985). Furthermore, techniques developed for the production of “chimeric antibodies” (Morrison et al., 1984; Neuberger et al., 1984; Takeda et al., 1985) by splicing the genes from a mouse antibody molecule specific for epitopes of SLLP polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

Various techniques have been developed for the production of antibody fragments of humanized antibodies. Traditionally, these fragments were derived via proteolytic digestion of full-length antibodies (see e.g., Morimoto & Inouye, 1992; Brennan et al., 1985). However, these fragments can now be produced directly by recombinant host cells. Alternatively, Fab′-SH fragments can be directly recovered from E. coli and chemically coupled to form F(ab′)2 fragments (Carter et al., 1992a). According to another approach, F(ab′)2 fragments can be isolated directly from recombinant host cell culture. Other techniques for the production of antibody fragments will be apparent to the skilled practitioner. See PCT International Patent Application Publication No. WO 1993/16185; U.S. Pat. Nos. 5,571,894; 5,587,458. The antibody fragment can also be a “linear antibody”, e.g., as described in U.S. Pat. No. 5,641,870, for example. Such linear antibody fragments can be monospecific or bispecific.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see e.g., U.S. Pat. Nos. 4,975,369; 5,075,431; 5,081,235; 5,169,939; 5,202,238; 5,204,244; 5,231,026; 5,292,867; 5,354,847; 5,472,693; 5,482,856; 5,491,088; 5,500,362; and 5,502,167). Detailed methods for preparation of chimeric (humanized) antibodies can be found in U.S. Pat. No. 5,482,856. A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see e.g., Jones et al., 1986; Riechmann et al., 1988; Presta, 1992, PCT International Patent Application Publication No. WO 92/02190, U.S. Patent Application Publication No. 2006/0073137, and U.S. Pat. Nos. 5,225,539; 5,530,101; 5,585,089; 5,693,761; 5,693,762; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; and 6,750,325.

In some embodiments, this presently disclosed subject matter provides for fully human antibodies. Human antibodies consist entirely of characteristically human polypeptide sequences. The human antibodies of this presently disclosed subject matter can be produced in using a wide variety of methods (see e.g., U.S. Pat. No. 5,001,065, for review).

Typically, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al, 1986; Riechmann et al., 1988); Verhoeyen et al., 1988), by substituting hypervariable region sequences for the corresponding sequences of a human “acceptor” antibody. Accordingly, such “humanized” antibodies are chimeric antibodies (see e.g., U.S. Pat. Nos. 4,816,567 and 5,482,856) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

Another method for making humanized antibodies is described in U.S. Patent Application Publication No. 2003/0017534, wherein humanized antibodies and antibody preparations are produced from transgenic non-human animals. The non-human animals are genetically engineered to contain one or more humanized immunoglobulin loci that are capable of undergoing gene rearrangement and gene conversion in the transgenic non-human animals to produce diversified humanized immunoglobulins.

In some embodiments, the choice of human variable domains, both light and heavy, to be used in making the humanized antibodies is very important to reduce antigenicity. According to the so-called “best-fit” method, the sequence of the variable domain of a rodent antibody is screened against a library of known human variable-domain sequences or a library of human germline sequences. The human sequence that is closest to that of the rodent can then be accepted as the human framework region for the humanized antibody (Sims et al., 1993; Chothia & Lesk, 1987). Another method uses a particular framework region derived from the consensus sequence of all human antibodies of a particular subgroup of light or heavy chains. The same framework can be used for several different humanized antibodies (Carter et al., 1992b; Presta et al., 1993). Other methods designed to reduce the immunogenicity of the antibody molecule in a human patient include veneered antibodies (see e.g., U.S. Pat. No. 6,797,492 and U.S. Patent Application Publication Nos. 2002/0034765 and 2004/0253645) and antibodies that have been modified by T-cell epitope analysis and removal (see e.g., U.S. Patent Application Publication No. 2003/0153043 and U.S. Pat. No. 5,712,120).

It is important that when antibodies are humanized they retain high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The antibody moieties of this presently disclosed subject matter can be single chain antibodies.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, and F(ab′)₂.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

Monoclonal antibodies directed against full length or peptide fragments of a protein or peptide can be prepared using any well known monoclonal antibody preparation procedures, such as those described, for example, in Harlow & Lane, 1988; Tuszynski et al., 1988). Quantities of the desired peptide can also be synthesized using chemical synthesis technology. Alternatively, DNA encoding the desired peptide can be cloned and expressed from an appropriate promoter sequence in cells suitable for the generation of large quantities of peptide. Monoclonal antibodies directed against the peptide are generated from mice immunized with the peptide using standard procedures as referenced herein.

Exemplary complementarity-determining region (CDR) residues or sequences and/or sites for amino acid substitutions in framework region (FR) of such humanized antibodies having improved properties such as, e.g., lower immunogenicity, improved antigen-binding or other functional properties, and/or improved physicochemical properties such as, e.g., better stability, are provided.

The presently disclosed subject matter encompasses more than the specific fragments and humanized fragments disclosed herein. In some embodiments, the antibody is selected from the group consisting of a single chain antibody, a monoclonal antibody, a bi-specific antibody, a chimeric antibody, a synthetic antibody, a polyclonal antibody, or a humanized antibody, or active fragments or homologs thereof.

A nucleic acid encoding an antibody obtained using the procedures described herein can be cloned and sequenced using technology that is available in the art, and is described, for example, in Wright et al., 1992 and the references cited therein. Further, the antibody of the presently disclosed subject matter can be “humanized” using the technology described in Wright et al., 1992 and in the references cited therein, and in Gu et al., 1997.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Green & Sambrook, 2012.

Bacteriophage which encode the desired antibody, can be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton & Barbas, 1994). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the presently disclosed subject matter should not be construed to be limited solely to the generation of phage encoding Fab antibodies. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. 20) Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions can be synthesized such that they include nearly all possible specificities (Barbas, 1995; de Kruif et al., 1995).

In the production of antibodies, screening for the desired antibody can be accomplished by techniques known in the art, e.g., ELISA (enzyme-linked immunosorbent assay). Antibodies generated in accordance with the presently disclosed subject matter can include, but are not limited to, polyclonal, monoclonal, chimeric (i.e., “humanized”), and single chain (recombinant) antibodies, Fab fragments, and fragments produced by a Fab expression library.

In some embodiments, the presently disclosed antibodies, fragments, and variants thereof can be purified after expression in E. coli. By way of example and not limitation, E. coli can be used as a host for recombinant protein production, including immunoglobulin fragments, as can mammalian cells. E. coli can be employed to produce the antibodies and fragments of the presently disclosed subject matter in large quantities (see e.g., Verma et al., 1998).

In some embodiments, the E. coli strain used can be BL21 DE3, which expresses T7 polymerase. This polymerase transcribes DNA sequences up to five times faster than the native E. coli RNA polymerase and enables strong induction protein expression with isopropyl β-D-1-thiogalactopyranoside (IPTG). Additionally, BL21 DE3 lacks the Lon protease (Gottesman, 1996) and outer membrane protease OmpT (Grodberg & Dunn, 1988).

The presently disclosed antibodies, fragments, and derivatives thereof can be also purified via affinity HPLC using a protein L column, which preferentially binds antibody light chains.

Substantially pure peptide obtained as described herein can be purified by following known procedures for protein purification, wherein an immunological, enzymatic, or other assay is used to monitor purification at each stage in the procedure. Protein purification methods are well known in the art, and are described, for example in Deutscher et al., 1990.

It is common in the field of recombinant humanized antibodies to graft murine CDR sequences onto a well-established human immunoglobulin framework previously used in human therapies such as the framework regions of Herceptin (Trastuzumab). In some embodiments, an expression cassette encoding one or more of heavy chain framework regions 1-4, including but not limited to those set forth as SEQ ID NOs: 40-43, respectively, and/or light chain framework regions 1-4, including but not limited to those set forth as SEQ ID NOs: 44-47, respectively, is employed. Into such an expression cassette, nucleotide sequences encoding the CDRs disclosed herein (i.e., SEQ ID NOs: 7, 9, and 11 encoding SEQ ID NOs: 8, 10, and 12 for heavy chain CDRs 1-3, respectively, and SEQ ID NOs: 19, 21, and 23 encoding SEQ ID NOs: 20, 22, and 24 for light chain CDRs 1-3, respectively) can be introduced, such that full length heavy and light chain variable regions, including but not limited to those encoded by SEQ ID NOs: 3 (encoding SEQ ID NO: 4) and SEQ ID NO: 15 (encoding SEQ ID NO: 16), respectively, can be produced in host cells.

In some embodiments, when used in vivo for therapy, the antibodies or the fragments and/or derivatives thereof of the subject presently disclosed subject matter are administered to the subject in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks. Optionally, the administration is made during the course of adjunct therapy such as antimicrobial treatment, or administration of tumor necrosis factor, interferon, or other cytoprotective or immunomodulatory agent.

In some embodiments, for parenteral administration, the antibodies or the fragments and/or derivatives thereof can be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

### Pharmaceutical Compositions and Administration

The presently disclosed subject matter is also directed to methods of administering the antibodies, fragments, and/or derivatives thereof of the presently disclosed subject matter to a subject.

Pharmaceutical compositions comprising the presently disclosed antibodies, fragments, and/or derivatives thereof are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with some embodiments, a method of treating a subject in need of such treatment is provided. The method comprises administering a pharmaceutical composition comprising at least one antibody, fragment, and/or derivative thereof of the presently disclosed subject matter to a subject in need thereof. Antibodies, fragments, and/or derivatives thereof of the presently disclosed subject matter can be administered with other known biologically active agents and/or other medications as well.

The pharmaceutical compositions useful for practicing the presently disclosed subject matter can be administered to deliver a dose of between 1 ng/kg/day and 100 mg/kg/day.

The presently disclosed subject matter encompasses the preparation and use of pharmaceutical compositions comprising at least one antibody, fragment, and/or derivative thereof useful for treatment of the diseases and disorders disclosed herein as an active ingredient. Such a pharmaceutical composition can consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter can comprise at least one active antibody, fragment, and/or derivative thereof, one or more acceptable carriers, and optionally other antibodies, fragments, and/or derivatives thereof and/or therapeutic agents.

For in vivo applications, the antibodies, fragments, and/or derivatives thereof of the presently disclosed subject matter can comprise a pharmaceutically acceptable salt. Suitable acids which are capable of forming such salts with the antibodies, fragments, and/or derivatives thereof of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, and/or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The antibodies, fragments, and/or derivatives thereof of the presently disclosed subject matter, pharmaceutically acceptable salts thereof, or pharmaceutical compositions comprising the same can be administered so that the antibodies, fragments, and/or derivatives thereof provide a desirable physiological effect. Administration can occur enterally or parenterally; for example, orally, rectally, intracisternally, intravaginally, intraperitoneally, locally (e.g., with powders, ointments, or drops), or as a buccal or nasal spray or aerosol. In some embodiments, the administration is parenteral. Exemplary parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, and direct application to the target area, for example by a catheter or other placement device.

Where the administration of an antibody, fragment, and/or derivative thereof is by injection or direct application, the injection or direct application can be in a single dose or in multiple doses. Where the administration of the antibody, fragment, and/or derivative thereof is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient.

In addition to the active ingredient, a pharmaceutical composition of the presently disclosed subject matter can further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which can be included in the pharmaceutical compositions of the presently disclosed subject matter are known in the art and described, for example in Genaro, 1985, which is incorporated herein by reference.

Typically, dosages of the compound of the presently disclosed subject matter which can be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, can also be prepared. The preparation can also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation can also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes adventitially administering the composition to a cell or a tissue of a subject. In some embodiments, this kit comprises a (in some embodiments sterile) solvent suitable for dissolving or suspending the composition of the presently disclosed subject matter prior to administering the compound to the subject.

### Idiopathic Pulmonary Fibrosis

Fibronectin (Fn) has been identified as a potential target for early onset Idiopathic Pulmonary Fibrosis (IPF). IPF produces similar scarring and thickening of tissue found in other forms of respiratory diseases, making it difficult to identify the exact cause of the disease. A possible pathway in the development of fibrosis involves a strained conformation of Fn. The presently disclosed subject matter provides an antibody called H5 to bind to the disease-state model, which is a strained conformation of Fn, called FnIII9-4G-10 (4G). Respresentative antibodies that bind better to 4G than H5 does are also provided herein. By way of example and not limitation, mutations were introduced into the H5 sequence by mutagenesis and amplified through error prone PCR to develop a diverse library of antibodies. This library was then screened using phage display and multiple ELISA rounds. Clone strength was quantified through an absorbance ratio, where higher ratios indicated better binding performance. Testing helped to identify 23 clones that outperformed H5; however, when the clones were sequenced, four were identified to be different than H5.

Pulmonary fibrosis (PF) is a disease in which lung tissue becomes thick, stiff, and scarred over time. Scarred lung tissue hinders the movement of oxygen from the lungs into the bloodstream, thus reducing the amount of available oxygen in circulation for the body (see e.g., the website of the National Heart, Lung, and Blood Institute; King et al., 2011). PF is further characterized by the deposition of extracellular matrix (ECM) proteins, such as fibronectin (Fn), that are responsible for the breakdown of functional alveolar units, which results in respiratory failure (Datta et al., 2011). While a controllable amount of ECM production benefits the body, such as through scar tissue for injury healing, an uncontrollable amount can be fatal, such as in Idiopathic Pulmonary Fibrosis (IPF; Raghu & Mikacenic, 2018). IPF, one of at least 200 different forms of PF, is a chronic state of fibrosis that leads to an irreversible decline in lung functionality. IPF affects 1 out of 200 adults over the age of 65, with 50,000 adults diagnosed and another 40,000 deaths from IPF each year (Pulmonary Fibrosis Foundation, 2018). Immediate treatment is required in order to slow the progression of IPF. Therefore, it is imperative to continue research to better detect and understand the pathways of IPF.

The presently disclosed representative H5 antibody binds to the strained conformation of Fn. Two different fragments were designed to model the strained and normal conformations in the relevant region of Fn. The strained version of Fn, FnIII9-4G-10 (4G), includes the addition of four glycines between the 9th and 10th type III repeats, which is a mutation that decreases binding affinity of Fn for the a5B1 integrin. The engineered Fn was used in experiments to model the change in response to mechanical forces in disease-states and to prove the existence of Fn's integrin switch. The normal version, FnIII9*10 (9*10), expresses the normal folding of Fn in the 9th and 10th type III repeats. Thus, modifications of the H5 antibody to improve binding to strained Fn and prohibit or limit binding to regular Fn are provided in accordance with the presently disclosed subject matter through directed evolution, phage display, and ELISAs.

As disclosed herein, the exemplary H5 antibody of the presently disclosed subject matter has been identified to possess a “local maximum” binding affinity for 4G. However, the binding has not reached the “absolute maximum”, or the best potential binding state. The basis for directed evolution is the production of a library with a maximal diversity of genes in order to reach fitness peaks. Therefore, also disclosed herein are experiments to develop a library of H5 antibody clones diverse enough to reach the “absolute maximum” binding affinity for 4G. In some embodiments, random mutagenesis is employed to introduce random mutations into the current H5 antibody DNA sequence (Cadwell & Joyce, 1992).

Phage display technology is used to improve the H5 clone's binding affinity for 4G and prohibit or limit binding to 9*10. Phage display is a process used to present polypeptides on the surface of lysogenic filamentous bacteriophages through manipulation of the phage's genotype (Bazan et al., 2012). Its use in creating antibodies was developed within a larger framework called directed evolution, which uses a trial-and-error approach to introduce genetic variations in antibodies in order to enhance at least one antibody's target binding ability (Trafton, 2010). The technique earned publicity recently when its contributors received the Nobel Prize in Chemistry (Offord & Grens, 2018). Through this framework, in some embodiments provided are antibodies and antigen-binding fragments thereof that are modified through optimization of the diversity of the antibody library by mutagenesis through error prone PCR, while also confirming that protein adaptations can be successful and be produced on a faster time scale.

Finally, ELISAs were performed (see EXAMPLE 1), and the clones identified to outperform H5 were separated from the phage for further antibody characterization. Sequences of the clones were analyzed as disclosed herein.

### EXAMPLE

The following EXAMPLE provides an illustrative embodiment. In light of the present disclosure and the general level of skill in the art, those of skill will appreciate that the following EXAMPLE is intended to be exemplary only and that numerous changes, modifications, and alterations can be employed without departing from the scope of the presently disclosed subject matter.

### Example 1

ELISA Analyses of Various Exemplary Antibodies and scFvs

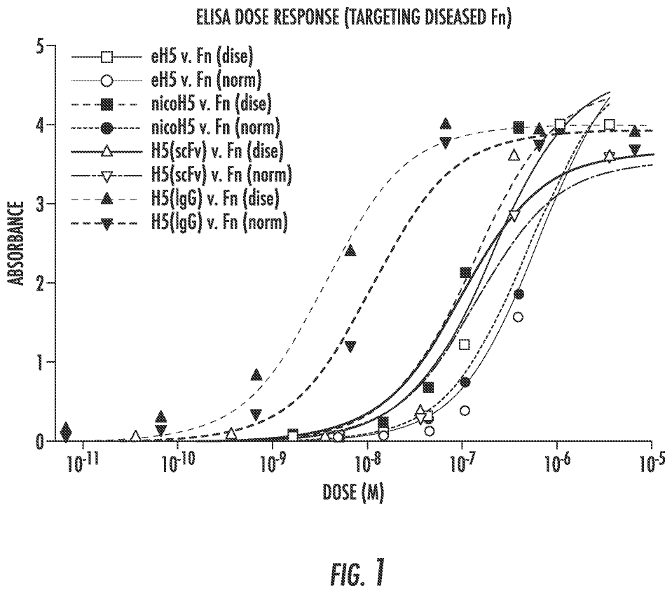

An unmodified H5 antibody was produced in either E. coli (depicted as eH5 and H5 (scFv) in FIG. 1) or Nicotiana benthamiana (nicoH5). The modified H5-IgG1 (H5-IgG) antibody of the presently disclosed subject matter was produced in a mammalian hybridoma cell line known as a plug-and-(dis)play hybridoma (PnP).

In vitro ELISA binding assays were employed to determine the efficacy of binding of H5 antibodies and derivatives thereof to the diseased form (dise) and the healthy form (norm) of fibronectin. Maleimide activated ELISA plates were used to tether the recombinant fibronectin fragments, which were named as FN-4G-10 and FN-9*-10, and they mimicked diseased and normal form of FN, respectively. Both formats of H5 were used in different molar concentrations starting with 10-5M and ending with 10-11M. Bound antibodies were detected by colorimetric method.

The results are shown in FIG. 1. The data represents the combination of two independent experiments, one comparing E. coli (eH5) versus N. benthamiana (nicoH5) produced H5-scFv and the second comparing H5-scFv (scFv) to H5-IgG1 (H5-IgG). Between the two formats of H5 tested, the modified IgG1 format of H5 demonstrated a much higher affinity for the FN fragments, showing at least 50-fold higher binding when compared with equimolar concentration of antibodies were used. Further, it discriminated the two fragments showing higher affinity for FN-4G-10 (diseased form) compared to FN-9*-10 (Healthy form).

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Addgene (2017) Over-Agar Antibiotic Plating. Basic Molecular Biology Protocol available from the website of Addgene of Watertown, Massachusetts, United States of America.
Altschul et al. (1990a) J Mol Biol 215:403-410.
Altschul et al. (1990b) Proc Natl Acad Sci USA 87:14:5509-5513.
Altschul et al. (1997) Nucleic Acids Res 25:3389-3402.
Arimori et al. (2017) Structure 25:1611-1622.
Aota et al. (1994) J Biol Chem 269:24756-247561.
Barbas (1995) Nature Medicine 1:837-839.
Bazan et al. (2012) Hum Vaccines Immunother 8:1817-1828.
Benjamin & Perdue (1996) Methods 9:508-515.
Bird et al. (1988) Science 242:423-426.
Bodanszky & Bodanszky (1984) in The Practice of Peptide Synthesis, Springer-Verlag, New York, New York, United States of America.
Brennan et al. (1985) Science 229:81-83.
Brockmann (2012) Selection of Stable scFv Antibodies by Phage Display. in Antibody
Engineering: Methods and Protocols, Second Edition (Chames (ed.)) Humana Press, New York, New York, United States of America, pages 123-144.
Brown et al. (2011) Tissue Eng, Part A 17:139-50.
Brown et al. (2015) J Biol Chem 290:25534-25547.
Burton & Barbas (1994) Adv Immunol 57:191-280.
Cadwell & Joyce (1992) PCR Methods Appl 2:28-33.
Cao et al. (2012) Proc Natl Acad Sci USA 109:7251-7256.
Cao et al. (2017) ACS Nano 11:7110-7117.
Carisey et al. (2013) Curr Biol 23:271-281.
Carter et al. (1992a) Bio/Technology 10:163-167.
Carter et al. (1992b) Proc Natl Acad Sci USA 89:4285.
Chandler et al. (2011) Phys Biol 8:015008.
Chou & Fasman (1974) Biochemistry 13:222-245.
Chou & Fasman (1978) Ann Rev Biochem 47:251-276.
Chou & Fasman (1979) Biophys J 26:367-384.
Chen et al. (2013) Adv Drug Deliv Rev 65:1357-1369.
Chothia & Lesk (1987) J Mol Biol 196:901-917.
Cole et al. (1985) in Monoclonal Antibodies and Cancer Therapy, Alan R. Liss, Inc., New York, New York, United States of America, pp. 77-96.
Cote et al. (1983) Proc Natl Acad Sci USA 80:2026-2030.
Craig et al. (2001) Proc Natl Acad Sci USA 98:5590-5595.
Craig et al. (2004) Structure 2004, 12, 21-30.
Craig et al. (2008) Langmuir 24:10282-10292.
Cuccuru et al. (2012) J Immunol Methods 382:216-219.
Datta et al. (2011) Br J Pharmacol 163:141-172.
de Kruif et al. (1995) J Mol Biol 248:97-105.
de Wildt et al. (2000) Nat Biotechnol 18:989-994.
Deutscher et al. (1990) Guide to Protein Purification, Harcourt Brace Jovanovich, San Diego, California, United States of America.
Devereux et al. (1984) Nucl Acids Res 12:387-395.
European Patent Application No. 17868682.0.
Foote & Winter (1992) J Mol Biol 224:487-499.
Gallant et al. (2005) Mol Biol Cell 16(9):4329-4340.
Garcia et al. (2002) Biochemistry 41:9063-9069.
Gee et al. (2008) PLOS One 3:e2373.
Genaro (1985) Remington's Pharmaceutical Sciences, Mack Publishing Co., Easton, Pennsylvania, United States of America.
Gibson (2011) Methods in Enzymology 498:349-361.
Gottesman (1996). Annual Review of Genetics, 30(1):465-506.
Grashoff et al. (2010) Nature 466:263-266.
Green & Sambrook (2012) Molecular Cloning: A Laboratory Manual, Fourth Edition, Cold Spring Harbor Laboratory Press, Cold Spring Harbor, New York, United States of America.
Grodberg & Dunn (1988) J Bacteriol 170:1245-1258.
Gross & Mienhofer (1981) The Peptides, Vol. 3, Academic Press, New York, New York, United States of America, pp. 3-88.
Gu et al. (1997) Thromb Haemost 77(4): 755-759.
Harlow & Lane (1988) Antibodies, A Laboratory Manual, Cold Spring Harbor Laboratory Publications, Cold Spring Harbor, New York, United States of America.
Henderson et al. (2013) Nat Med 19:1617-1624.
Hubbard et al. (2016) J Cell Physiol 231:1728-1736.
Huse et al. (1989) Science 246:1275-1281.
Huston et al. (1988) Proc Natl Acad Sci USA 85:5879-5883.
Izbicki et al. (2002) Int J Exp Pathol 83:111-119.
Jiang et al. (1994) J Cell Sci 107:2499-2508.
Jones et al. (1986) Nature 321:522-525.
Karlin & Altschul (1990) Proc Natl Acad Sci USA 87:2264-2268.
King et al. (2011) Lancet 378(9807): 1949-1961.
Karlin & Altschul (1993) Proc Natl Acad Sci USA 90:5873-5877.
Koenig et al. (2017) Proc Natl Acad Sci USA 114:486-495.
Kohler & Milstein (1975) Nature 256:495-497.
Kozbor & Roder (1983) Immunology Today 4:72.
Krammer et al. (1999) Proc Natl Acad Sci USA 96:1351-1356.
Kuddannaya et al. (2013) ACS Appl Mater Interfaces 5:9777-9784.
Kyte & Doolittle (1982) J Mol Biol 157:105-132.
Lawson et al. (2005) Am J Pathol 167:1267-1277.
Lee et al. (2007) Nat Protoc 2:3001-3008.
Lemmon et al. (2011) J Biol Chem 286:26375-26382.
Li et al. (2005) J Mol Biol 345:817-826.
Little et al. (2008) Matrix Biol 27:451-461.
Mardon & Grant (1994) FEBS Lett 340:197-201.
Markowski et al. (2012) J Biomed Mater Res, Part A 100:2119-2127.
Marks et al. (1991) J Mol Biol 222:581-597.
Martino et al. (2009) Biomaterials 30:1089-1097.
Moek et al. (2017) J Nucl Med Off Publ Soc Nucl Med 58:83S-90S.
Morimoto & Inouye (1992) J Biochem Biophys Methods 24:107-117.
Morrison et al. (1984) Proc Natl Acad Sci USA. 81:6851-6855.
Morton & Myszka (1998) Methods Enzymol 295:268-294.
Morton et al. (1995) Anal Biochem 227(1):176-185.
Mould et al. (1997) J Biol Chem 272:17283-17292.
Myszka et al. (1998) Trends Biochem Sci 23(4):149-150.
National Heart, Lung, and Blood Institute. “Idiopathic Pulmonary Fibrosis”, available from the website of the National Heart, Lung, and Blood Institute (NHLBI).
Neuberger et al. (1984) Nature 312:604-608.
Offord & Grens (2018) The Scientist Magazine, Oct. 3, 3018.
Pankov & Yamada (2002) J Cell Sci 115:3861-3863.
Patan (2004) Cancer Treat Res 117:3-32.
Paul (1993) Fundamental Immunology, 3rd Ed., Raven Press, New York, New York, United States of America.
PCT International Patent Application Publication Nos. WO 1992/02190; WO 1993/16185; WO 2007/019107; WO 2007/030652; WO 2007/089798; WO 2008/060374; WO 2018/088403.
Pitulescu et al. (2010) Nat Protoc 5:1518-1534.
Presta (1992) Curr Op Struct Biol 2:593-596.
Presta et al. (1993) J Immunol 1993 151:2623.
Pulmonary Fibrosis Foundation (2018) What is Pulmonary Fibrosis. available from the website of the Pulmonary Fibrosis Foundation, Chicago, Illinois, United States of America.
Raghu & Mikacenic (2018) Pathogenesis of idiopathic pulmonary fibrosis. Available from the website of UPTODATE®, Wolters Kluwer, Riverwoods, Illinois, United States of America.
Riechmann et al. (1988) Nature 332:323-327.
Rosano & Ceccarelli (2014) Front Microbiol 5:Article 172.
Ruoslahti & Pierschbacher (1987) Science 238:491-497.
Schornberg et al. (2009) Proc Natl Acad Sci USA 106:8003-8008.
Shukla et al. (2013) Nature 497:137-141.
Sidhu (2001) Biomol Eng 18:57-63.
Sims et al. (1993) J Immunol 151:2296-2308.
Skerra & Pluckthun (1988). Science 240:1038.
Smith et al. (2007) PLOS Biol 5:2243-2254.
Stewart et al. (1984) in Solid Phase Peptide Synthesis, 2nd Edition, Pierce Chemical Company, Rockford, Illinois, United States of America.
Takeda et al. (1985) Nature 314:452-454.
Tobias (2014) Forte Bio Appl Note 14:1-21.
Trafton (2010) MIT News May 13, 2010. Massachusetts Institute of Technology, Cambridge, Massachusetts, United States of America.
Tuszynski et al. (1988) Blood 72:109-115.
U.S. patent application Ser. No. 16/345,639.
U.S. Patent Application Publication Nos. 2002/0034765; 2003/0017534; 2003/0022244; 2003/0153043; 2004/0253645; 2006/0073137; 2018/0298087, 2018/0312588, 2018/0346564, 2019/0151448.
U.S. Pat. Nos. 4,554,101; 4,816,567; 4,946,778; 4,975,369; 5,001,065; 5,075,431; 5,081,235; 5,169,939; 5,202,238; 5,204,244; 5,225,539; 5,231,026; 5,292,867; 5,354,847; 5,436,157; 5,472,693; 5,482,856; 5,491,088; 5,500,362; 5,502,167; 5,530,101; 5,571,894; 5,585,089; 5,587,458; 5,641,870; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,479,284; 6,548,640; 6,632,927; 6,639,055; 6,677,436; 6,750,325; 6,797,492; 7,060,808; 7,906,625; 8,398,980; 8,436,150; 8,796,439; 10,253,111.
van der Walle et al. (2002) Protein Eng 15:1021-1024.
Verhoeyen et al. (1988) Science 239:1534-1536.
Verma et al. (1998). Journal of Immunological Methods 216(1-2), 165-181.
Winter & Milstein (1991) Nature 349:293-299.
Wright et al. (1992) Critical Rev in Immunol 12(3,4):125-168.
Zhu et al. (2008) Mol Cell 32:849-861.

While the presently disclosed subject matter has been disclosed with reference to specific embodiments, it is apparent that other embodiments and variations of this presently disclosed subject matter can be devised by others skilled in the art without departing from the true spirit and scope of the presently disclosed subject matter.

1. An isolated and purified antibody comprising an amino acid sequence selected from the group consisting of SEQ ID NOs: 2, 4, 6, 14, 16, or 18, a fragment thereof comprising a paratope, or an antibody having an amino acid sequence that is approximately 95% identical to the sequence of any one of SEQ ID NOs: 2, 4, 6, 14, 16, or 18, or a fragment thereof, wherein:
(i) the isolated and purified antibody comprises a heavy chain CDR1 amino acid sequence comprising SYAMS (SEQ ID NO: 8), a heavy chain CDR2 amino acid sequence comprising DIYDGGGTNYADSVKG (SEQ ID NO: 10), a heavy chain CDR3 amino acid sequence comprising TADNFDY (SEQ ID NO: 12), a light chain CDR1 amino acid sequence comprising RASQSISSYLN (SEQ ID NO: 20), a light chain CDR2 amino acid sequence comprising AASTLOS (SEQ ID NO: 22), and a light chain CDR3 amino acid sequence comprising QQANSAPTT (SEQ ID NO: 24); and/or
(ii) the isolated and purified antibody further comprises a heavy chain framework region 1 comprising EVQLLESGGGLVQPGGSLRLSCAAS (SEQ ID NO: 40), a heavy chain framework region 2 comprising WRQAPGKGLEWV (SEQ ID NO: 41), a heavy chain framework region 3 comprising RFTTSRDNSKNTLYLQMNSLRAEDTAVYYC (SEQ ID NO: 42), and a heavy chain framework region 4 comprising WGQGTLVTVSS (SEQ ID NO: 43); and/or
(iii) the isolated and purified antibody further comprises a light chain framework region 1 comprising DIQMTQSPSSLSASVGDRVTITC (SEQ ID NO: 44), a light chain framework region 2 comprising WYQQKPGKAPKLLIY (SEQ ID NO: 45), a light chain framework region 3 comprising GVPSRFSGSGSGTDFTLTISSLQPEDFATYYC (SEQ ID NO: 46), and a light chain framework region 4 comprising FGQGTKVEIK (SEQ ID NO: 47).

2. The isolated and purified antibody of claim 1, wherein the amino acid sequence comprises at least one modification selected from the group consisting of an amino acid deletion, an amino acid addition, an amino acid substitution, and combinations thereof.

3. The isolated and purified antibody of claim 1, wherein the antibody is humanized.

4. An isolated and purified nucleic acid sequence encoding the antibody of claim 1, or a fragment thereof comprising a paratope.

5. A method for targeting a conformational state of fibronectin (FN) in a sample, optionally a biological sample isolated from or present within a subject, the method comprising contacting the sample with the isolated and purified antibody of claim 1, or a paratope-containing fragment thereof.

6. The method of claim 5, wherein the sample comprises or is suspected to comprise a tissue undergoing tissue repair, a tissue that is diseased, a tissue that suffers from a disorder, or any combination thereof.

7. A method for detecting a conformational state of fibronectin (FN) in a sample, the method comprising contacting the sample with the isolated and purified antibody of claim 1, or a paratope-containing fragment thereof; and detecting the binding of the composition, whereby the conformational state of FN is detected.

8. The method of claim 7, wherein the sample comprises or is suspected to comprise a tissue undergoing tissue repair, a tissue that is diseased, a tissue that suffers from a disorder, or a combination thereof.

9. The method of claim 7, wherein the sample comprises or is suspected to comprise a pathologic extracellular matrix (ECM).

10. The method of claim 7, wherein the sample comprises or is suspected to comprise a fibrotic ECM.

11. The method of claim 7, wherein detecting the binding of the composition comprises detecting a binding ratio of composition to FN.

12. The method of claim 7, wherein detecting the binding of the composition comprises distinguishing normal from diseased tissue.

13. The method of claim 7, wherein detecting the binding of the composition comprises determining severity of fibrosis in the sample.

14. The method of claim 7, wherein detecting the binding of the composition comprises detecting a transient, force-induced conformational change in FN.

15. The method of claim 7, wherein detecting the binding of the composition comprises extracting structural information for an ECM in the sample.

16. The method of claim 15, wherein extracting structural information for an ECM in the sample comprises delineating regions of high ECM strain.

17. The method of claim 16, wherein the high ECM strain is associated with enhanced αv integrin binding character.

18. The method of claim 7, further comprising determining a type of treatment to be administered to the subject based on the detecting of the binding of the composition.

19. A method for treating a disease or disorder in a subject, the method comprising administering to a subject in need thereof the isolated and purified antibody of claim 1, or a paratope-containing fragment thereof, in an amount and via a route sufficient to treat the disease or the disorder in the subject.

20. The method of claim 19, wherein the disease or disorder has a characteristic selected from the group consisting of a tissue undergoing tissue repair, a tissue that is diseased, a tissue that suffers from a disorder, and any combination thereof.

21. The method of claim 20, wherein the characteristic is a pathologic extracellular matrix (ECM).

22. The method of claim 20, wherein the characteristic is a fibrotic ECM.

23. A method for ameliorating at least one symptom of consequence of a disease or disorder associated with abnormal expression of a force-induced conformational state of FN comprising FnIII9-4G-10 (4G) in a subject, the method comprising administering to a subject in need thereof the isolated and purified antibody of claim 1, or a paratope-containing fragment thereof, in an amount and via a route sufficient to amerliorate at least one symptom of the disease or the disorder in the subject, wherein at least one symptom of consequence of a disease or disorder associated with abnormal expression of a force-induced conformational state of FN comprising FnIII9-4G-10 (4G) is ameliorated.

24. The method of claim 23, wherein the disease or disorder is associated with a tissue undergoing tissue repair, a tissue that is diseased, a tissue that suffers from a disorder, or any combination thereof.

25. The method of claim 23, wherein the disease or disorder is associated with a pathologic extracellular matrix (ECM).

26. The method of claim 23, wherein the disease or disorder is associated with a fibrotic ECM.

27. The isolated and purified antibody of claim 1, wherein the antibody further comprises a modification at its N-terminus, its C-terminus, or both.

28. The isolated and purified antibody of claim 27, wherein the modification comprises addition of a peptide tag, a SARAH domain, or a combination thereof.

29. The isolated and purified antibody of claim 28, wherein the tag comprises a his tag, a myc tag, a VSV tag, an HA tag, a SortaseA tag, a PelB sequence, or any combination of one or more thereof.

30. The isolated and purified antibody of claim 28, wherein the SARAH domain comprises a sequence selected from the group consisting of SEQ ID NOs: 28-32.


SEQUENCE LISTING (49 sequences)

SEQ ID NO: 1 - DNA, 1337 bp, Artificial Sequence
  Artififically synthesized sequence
aggtgcagttgctcgaatcaggtggtggactagtacaaccaggtggttctctccgactttcttgtgctgcatcagggtttacattttcttcctatgcgatgtcctgggtccgccaagcacccggcaaaggactagaatgggttagtgatatttatgatggggggggaacaaattatgcagatagtgtcaaaggacgctttacgacgtcccgggacaatagtaagaatacactttacttgcaaatgaattccctgcgcgctgaagatactgctgtctattattgtgcgaaaactgctgataattttgattattggggacaaggcaccctggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 2 - protein, 445 aa, Artificial Sequence
  Artififically synthesized sequence
EVQLLESGGGLVQPGGSLRLSCAASGFTFSSYAMSWVRQAPGKGLEWVSDIYDGGGTNYADSVKGRFTTSRDNSKNTLYLQMNSLRAEDTAVYYCAKTADNFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 344 bp, Artificial Sequence
  Artififically synthesized sequence
aggtgcagttgctcgaatcaggtggtggactagtacaaccaggtggttctctccgactttcttgtgctgcatcagggtttacattttcttcctatgcgatgtcctgggtccgccaagcacccggcaaaggactagaatgggttagtgatatttatgatggggggggaacaaattatgcagatagtgtcaaaggacgctttacgacgtcccgggacaatagtaagaatacactttacttgcaaatgaattccctgcgcgctgaagatactgctgtctattattgtgcgaaaactgctgataattttgattattggggacaaggcaccctggtcaccgtctcctca

SEQ ID NO: 4 - protein, 115 aa, Artificial Sequence
  Artififically synthesized sequence
EVQLLESGGGLVQPGGSLRLSCAASGFTFSSYAMSWVRQAPGKGLEWVSDIYDGGGTNYADSVKGRFTTSRDNSKNTLYLQMNSLRAEDTAVYYCAKTADNFDYWGQGTLVTVSS

SEQ ID NO: 5 - DNA, 993 bp, Artificial Sequence
  Artififically synthesized sequence
gcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 330 aa, Artificial Sequence [sequence omitted]

SEQ ID NO: 7 - DNA, 15 bp, Artificial Sequence
  Artififically synthesized sequence
tcctatgcgatgtcc

SEQ ID NO: 8 - protein, 5 aa, Artificial Sequence
  Artififically synthesized sequence
SYAMS

SEQ ID NO: 9 - DNA, 48 bp, Artificial Sequence
  Artififically synthesized sequence
gatatttatgatggggggggaacaaattatgcagatagtgtcaaagga

SEQ ID NO: 10 - protein, 16 aa, Artificial Sequence
  Artififically synthesized sequence
DIYDGGGTNYADSVKG

SEQ ID NO: 11 - DNA, 21 bp, Artificial Sequence
  Artififically synthesized sequence
actgctgataattttgattat

SEQ ID NO: 12 - protein, 7 aa, Artificial Sequence
  Artififically synthesized sequence
TADNFDY

SEQ ID NO: 13 - DNA, 645 bp, Artificial Sequence
  Artififically synthesized sequence
gacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcaccctgcagagcggcgtgcccagcaggttcagcggcagcggcagcggcaccgacttcaccctgaccatcagcagcctgcagcccgaggacttcgccacctactactgccagcaggccaacagcgcccccaccaccttcggccagggcaccaaggtggagatcaagcggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 14 - protein, 214 aa, Artificial Sequence
  Artififically synthesized sequence
DIQMTQSPSSLSASVGDRVTITCRASQSISSYLNWYQQKPGKAPKLLIYAASTLQSGVPSRFSGSGSGTDFTLTISSLQPEDFATYYCQQANSAPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 324 bp, Artificial Sequence
  Artififically synthesized sequence
gacatccagatgacccagagccccagcagcctgagcgccagcgtgggcgacagggtgaccatcacctgcagggccagccagagcatcagcagctacctgaactggtaccagcagaagcccggcaaggcccccaagctgctgatctacgccgccagcaccctgcagagcggcgtgcccagcaggttcagcggcagcggcagcggcaccgacttcaccctgaccatcagcagcctgcagcccgaggacttcgccacctactactgccagcaggccaacagcgcccccaccaccttcggccagggcaccaaggtggagatcaagcgg

SEQ ID NO: 16 - protein, 108 aa, Artificial Sequence
  Artififically synthesized sequence
DIQMTQSPSSLSASVGDRVTITCRASQSISSYLNWYQQKPGKAPKLLIYAASTLQSGVPSRFSGSGSGTDFTLTISSLQPEDFATYYCQQANSAPTTFGQGTKVEIKR

SEQ ID NO: 17 - DNA, 321 bp, Artificial Sequence
  Artififically synthesized sequence
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 18 - protein, 106 aa, Artificial Sequence
  Artififically synthesized sequence
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 33 bp, Artificial Sequence
  Artififically synthesized sequence
agggccagccagagcatcagcagctacctgaac

SEQ ID NO: 20 - protein, 11 aa, Artificial Sequence
  Artififically synthesized sequence
RASQSISSYLN

SEQ ID NO: 21 - DNA, 18 bp, Artificial Sequence
  Artififically synthesized sequence
gccgccagcaccctgcag

SEQ ID NO: 22 - protein, 7 aa, Artificial Sequence
  Artififically synthesized sequence
AASTLQS

SEQ ID NO: 23 - DNA, 27 bp, Artificial Sequence
  Artififically synthesized sequence
cagcaggccaacagcgcccccaccacc

SEQ ID NO: 24 - protein, 9 aa, Artificial Sequence
  Artififically synthesized sequence
QQANSAPTT

SEQ ID NO: 25 - protein, 5 aa, Artificial Sequence
  Artififically synthesized sequence
PHSRN

SEQ ID NO: 26 - protein, 4 aa, Artificial Sequence
  Artififically synthesized sequence
GGGG

SEQ ID NO: 27 - protein, 5 aa, Artificial Sequence
  Artififically synthesized sequence
SGGGG

SEQ ID NO: 28 - protein, 49 aa, Artificial Sequence
  Artififically synthesized sequence
DYEFLKSWTVEDLQKRLLALDPMMEQEIEEIRQKYQSKRQPILDAIEAK

SEQ ID NO: 29 - protein, 49 aa, Artificial Sequence
  Artififically synthesized sequence
DFDFLKNLSLEELQMRLKALDPMMEREIEELRQRYTAKRQPILDAMDAK

SEQ ID NO: 30 - protein, 49 aa, Artificial Sequence
  Artififically synthesized sequence
GEVEWDAFSIPELQNFLTILEKEEQDKIQQVQKKYDKFRQKLEEALRES

SEQ ID NO: 31 - protein, 49 aa, Artificial Sequence
  Artififically synthesized sequence
GEVNWDAFSMPELHNFLRILQREEEEHLRQILQKYSYSRQKIQEALHAS

SEQ ID NO: 32 - protein, 49 aa, Artificial Sequence
  Artififically synthesized sequence
HILKWELFQLADLDTYQGMLKLLFMKELEQIVKMYEAYRQALLTELENR

SEQ ID NO: 33 - protein, 10 aa, Artificial Sequence
  Artififically synthesized sequence
EQKLISEEDL

SEQ ID NO: 34 - protein, 11 aa, Artificial Sequence
  Artififically synthesized sequence
YTDIEMNRLGK

SEQ ID NO: 35 - protein, 6 aa, Artificial Sequence
  Artififically synthesized sequence
HHHHHH

SEQ ID NO: 36 - protein, 9 aa, Artificial Sequence
  Artififically synthesized sequence
YPYDVPDYA

SEQ ID NO: 37 - protein, 6 aa, Artificial Sequence
  Artififically synthesized sequence
LPETGG

SEQ ID NO: 38 - protein, 22 aa, Artificial Sequence
  Artififically synthesized sequence
MKYLLPTAAAGLLLLAAQPAMA

SEQ ID NO: 39 - protein, 5 aa, Artificial Sequence
  Artificially synthesized sequence
NSAAH

SEQ ID NO: 40 - protein, 25 aa, Artificial Sequence
  Artificially synthesized framework region amino acid sequence
EVQLLESGGGLVQPGGSLRLSCAAS

SEQ ID NO: 41 - protein, 13 aa, Artificial Sequence
  Artificially synthesized framework region amino acid sequence
WVRQAPGKGLEWV

SEQ ID NO: 42 - protein, 30 aa, Artificial Sequence
  Artificially synthesized framework region amino acid sequence
RFTTSRDNSKNTLYLQMNSLRAEDTAVYYC

SEQ ID NO: 43 - protein, 11 aa, Artificial Sequence
  Artificially synthesized framework region amino acid sequence
WGQGTLVTVSS

SEQ ID NO: 44 - protein, 23 aa, Artificial Sequence
  Artificially synthesized framework region amino acid sequence
DIQMTQSPSSLSASVGDRVTITC

SEQ ID NO: 45 - protein, 15 aa, Artificial Sequence
  Artificially synthesized framework region amino acid sequence
WYQQKPGKAPKLLIY

SEQ ID NO: 46 - protein, 32 aa, Artificial Sequence
  Artificially synthesized framework region amino acid sequence
GVPSRFSGSGSGTDFTLTISSLQPEDFATYYC

SEQ ID NO: 47 - protein, 10 aa, Artificial Sequence
  Artificially synthesized framework region amino acid sequence
FGQGTKVEIK

SEQ ID NO: 48 - protein, 5 aa, Artificial Sequence
  Artificially synthesized peptide tag
  misc_feature (3)..(3): Xaa can be any naturally occurring amino acid
LPXTG

SEQ ID NO: 49 - protein, 22 aa, Artificial Sequence
  Artificially synthesized peptide tag
MKYLLPTAEAGLLLLLAAPQIA